(19) United States
(12) Patent Application Publication
(10) Pub. No.: US 2007/0015133 A1
(43) Pub. Date: Jan. 18, 2007

(54) Method for detecting and/or removing protein and/or peptide comprising a cross-beta structure from an aqueous solution comprising a protein

(75) Inventors: Martijn Frans Ben Gerard, Eemnes (NL); Barend Bouma, Houten (NL)

Correspondence Address: TRASK BRITT, P.O. BOX 2550, SALT LAKE CITY, UT, 84110, US

(73) Assignee: UMC Utrecht Holding B.V., Utrecht (NL)

(21) Appl. No.: 11/181,012

(22) Filed: Jul. 13, 2005

(51) Int. Cl. C12Q 1/00; G01N 33/00; C07K 14/47

(52) U.S. Cl. 435/4; 436/86; 530/350

(57) ABSTRACT

The invention relates to the field of aqueous solutions comprising a protein. More specifically, the invention relates to the detection and/or removal of conformationally altered proteins and/or peptides comprising a cross-β structure from an aqueous solution comprising a protein. The invention provides methods for detecting and/or removing proteins and/or peptides comprising a cross-β structure from an aqueous solution comprising a protein, the method comprising contacting the aqueous solution comprising a protein with at least one cross-β structure-binding compound resulting in a bound protein or peptide with cross-β structure. The invention further provides a aqueous solution comprising a protein obtainable by a method of the invention, and a kit for carrying out the methods of the invention.

### TECHNICAL FIELD

The invention relates to the field of aqueous solutions comprising a protein. More specifically, the invention relates to the detection and/or removal of conformationally altered proteins and/or peptides comprising a cross-β structure from an aqueous solution comprising a protein.

### BACKGROUND

A protein or peptide is generally exposed to environmental influences which alter the original conformation and are, therefore, detrimental to the protein or peptide. Such environmental influences, for example, comprise temperature, light, pressure, humidity, enzymatic and microbial processes, pH and osmolarity of the solution, etc.

### SUMMARY OF THE INVENTION

Disclosed is that partially unfolded and/or misfolded proteins or peptides that are, for example, proteolyzed, denatured, partially unfolded, glycated, oxidized, acetylated, multimerized or otherwise structurally altered, adopt a cross-β structure conformation. Further disclosed is that unwanted side effects and decreased specific activity are caused by proteins adopting a cross-β structure conformation. The presence of a cross-β structure is, therefore, indicative for degraded and/or denatured and/or multimerized protein, peptide and/or polypeptide.

The terms “unfolding,” “refolding” and “misfolding” relate to the three-dimensional structure of a protein or peptide. “Unfolding” means that the protein or peptide loses the three-dimensional structure and takes a linear arrangement. The term “refolding” relates to the coiling back into the original three-dimensional structure. By refolding, a protein can regain its native configuration, or an incorrect refolding can occur. The incorrect refolding is also called “misfolding.” During unfolding and refolding, the formation of cross-β structures can occur.

Disclosed herein are methods and means for detecting cross-β structures in proteins, peptides and/or polypeptides, preferably in an aqueous solution. The present invention also discloses methods for removing cross-β structures from proteins, and/or peptides, preferably in an aqueous solution. The methods of the invention are suitable for diminishing the unwanted side effects and the toxicity of proteins, peptides and/or polypeptides, and for increasing the specific activity of proteins, and/or peptides.

Generally, the specific activity of a protein, peptide and/or polypeptide in a solution is decreased after formation of cross-β structures. In addition, a cross-β structure in turn increases the formation of more cross-β structures in the protein and/or peptides, thereby increasing the degradation and/or denaturation and/or refolding and/or multimerization. Furthermore, the presence of a cross-β structure in a protein, peptide and/or polypeptide increases the risk of toxic and other unwanted side effects when the protein, peptide and/or polypeptide is administered to a subject, the subject being an animal or a human.

Many proteins, peptides or polypeptides used by man for different purposes are either derived from natural sources such as animals or plants, or they are synthesized or produced in vitro. Proteins, peptides or polypeptides are, for example, also used in the preparation of products like, food products, base products for the production of food, detergents, preferably detergents comprising enzymes, and/or cosmetic products. The proteins, peptides or polypeptides are also used for diagnostic purposes like, for example, antisera and antigen preparations. In yet another embodiment, proteins, peptides or polypeptides are used in analytical or biochemical chemistry, for example, as commercially available biochemical base compounds such as proteins and enzymes, preferably in purified form.

Quality aspects are of great concern with any production and/or purification process comprising a protein, peptide or polypeptide. Protein stability during production, purification and storage is, therefore, important to manufacturers and to customers. Yet, in spite of these concerns, a number of accepted treatments of proteins may alter the conformation of the protein and therefore, induce cross-β structures in the protein.

For example, one generally accepted way of stabilizing a protein is by freezing the protein below zero degrees Celsius. Freezing and thawing may severely affect the conformation of proteins, peptides and/or polypeptides. Another accepted method of preservation is lyophilization. With this method, a protein is freeze-dried by evaporation of the aqueous solution below zero degrees Celsius. Many proteins and/or peptides are stored and sold in a dry form as lyophilized protein. Reconstitution of lyophilized protein or peptide with a suitable aqueous solution is generally performed before the protein is used. Both the freezing and evaporation step, but also the reconstituting step, comprise risks for conformational changes of the protein and/or peptide and the formation of cross-β structures in the protein and/or peptide. It is, for example, known that lyophilized proteins comprise a higher β-sheet content than their solubilized counterparts, indicative for a refolding process due to the treatment.

A protein and/or peptide in this specification comprises any protein and/or peptide that is capable of forming a cross-β structure. Alteration of the protein and/or peptide comprises, for example, denaturation, proteolysis, acetylation, glycation, oxidation or unfolding of proteins.¹⁻⁴

An increasing body of evidence shows that the partial or complete unfolding of initially properly folded native proteins leads to the formation of toxic structures in proteins.¹⁻⁴ The invention further discloses that partial unfolding results in the formation of crossed structures in a protein and/or peptide.

A cross-β structure is defined as a part of a protein or peptide, or a part of an assembly of peptides and/or proteins, which comprises an ordered group of β-strands, typically a group of β-strands arranged in a β-sheet, in particular a group of stacked β-sheets. A typical form of stacked β-sheets is in a fibril-like structure in which the β-sheets may be stacked in either the direction of the axis of the fibril or perpendicular to the direction of the axis of the fibril. Of course, the term peptide is intended to include oligopeptides as well as polypeptides, and the term protein includes proteins with and without post-translational modifications, such as glycosylation and glycation. It also includes lipoproteins and complexes comprising proteins, such as protein-nucleic acid complexes (RNA and/or DNA), membrane-protein complexes, etc.

Different fluorescent light scattering profiles of amyloid dyes, such as, for example, Congo red or Thioflavin T in staining various amyloid-like aggregates indicate that different forms of cross-β structures occur. Cross-β structures are, for example, found in glycated proteins and in fibrils.⁵ Such fibrillar aggregates accumulate in various tissue types and are associated with a variety of degenerative diseases. The term “amyloid” is being used to describe fibrillar deposits (or plaques).⁶ In literature, an amyloid fibril is preferably defined as an aggregate that is stained by Congo red and/or Thioflavin T, that appears as fibrils under an electron microscope, and that contains an increased amount of β-sheet secondary structure.⁶ Additionally, the presence of β-sheet rich structures can be defined with X-ray fiber diffraction techniques and/or Fourier transform infrared spectroscopy. A common denominator of amyloid-like structures is the presence of the cross-β structure structural element. Peptides or proteins with amyloid-like structures are cytotoxic to cells.⁷⁻¹¹

Diseases characterized by amyloid are referred to as conformational diseases or amyloidoses and include, for example, Alzheimer's disease (AD), light-chain amyloidosis, type II diabetes and spongiform encephalopathies like, for example, Bovine Spongiform Encephalopathy (BSE) and Creutzfeldt-Jakob's disease.

In addition, deleterious effects of aggregated proteins are not solely mediated by amyloid fibrillar depositions of proteins, but also by soluble oligomers of aggregates with amyloid-like properties and by diffuse amorphous aggregates.^{7, 129, 13, 14} The recent finding that toxicity is an inherent property of misfolded proteins implies a common mechanism for conformational diseases.^{5, 7, 10}

The compounds listed in Table 1 and the proteins listed in Table 2 all bind to polypeptides with cross-β structure. In literature, this fold has been designated as protein aggregates, amorphous aggregates, amorphous deposit, tangles, (senile) plaques, amyloid, amyloid-like protein, amyloid oligomers, amyloidogenic deposits, cross-β structure, β-pleated sheet, cross-β spine, denatured protein, cross-β sheet, β-structure rich aggregates, infective aggregating form of a protein, unfolded protein, amyloid-like fold/conformation and perhaps alternatively. The common theme amongst all polypeptides that are ligands for one or more of the compounds listed in Tables 1 and 2, is the presence of a cross-β structure.

The compounds listed in Table 1 and 2 are considered to be only an example of the compounds known to day to bind to proteins or peptides with cross-β structures. The lists are thus non-limiting. More compounds are known today that bind to amyloid-like protein conformation and are thus functional equivalents of the compounds in Table 1, 2, or 3. For example, in Australian Patent AU2003214375 it is described that aggregates of prion protein, amyloid, and tau bind selectively to polyionic-binding agents such as dextran sulphate or pentosan (anionic), or to polyamine compounds such as poly (diallyldimethylammonium Chloride) (cationic). Compounds with specificity for proteins and peptides with cross-β structure listed in this patent and elsewhere are equally suitable for methods and devices disclosed in this patent application. Moreover, also any compound or protein related to the ones listed in Tables 1 and 2 are covered by the claims. For example, point mutants, fragments, recombinantly produced combinations of cross-β structure-binding domains and deletion and insertion mutants are part of the set of compounds as long as they are capable of binding to protein with a cross-β structure (i.e., as long as they are functional equivalents). In addition, any small molecule or protein that exhibits affinity for the cross-β structure can be used in any one of the methods and applications disclosed here.

The compounds listed in Table 3 are also considered to be part of the “Cross-β structure pathway,” and this consideration is based on literature data that indicates interactions of the listed molecules with compounds that likely comprise a cross-β structure but that have not been disclosed as such.

Generally, for the production of a protein and/or peptide, the protein and/or peptide is subjected to a number of processes like, for example, a synthesis process or an isolation process. Peptide synthesis processes are generally performed in a plant cell, a yeast cell or bacteria, or a cell of an animal. A protein and/or peptide manufacturing process also comprises coupling of chemical molecules to a peptide or protein. Further, the protein and/or peptide is subjected to an isolation procedure or a purification procedure, and/or a concentrating process, like, for example, the isolation of recombinant protein from a bacterial production cell, or purification by a physical, or a chemical, or an immunological isolation method, and/or a formulation and/or a storage process, including, for example, a lyophilization process and/or the addition of a suitable stabilizer, a diluent and/or an adjuvant.

Any one of these processes affects the folding of a protein and/or peptide. Quality control in a manufacturing process preferably aims at identifying and/or minimizing the deleterious effects of each process step for a protein and/or peptide, thereby increasing the activity of the composition in the final product or composition and/or decreasing the undesired side effects of the composition.

Alteration of a protein and/or peptide is generally detected by two methods. The first method comprises measuring the amount of a specific binding site of protein and/or peptide. The second method comprises measuring an increase in size or multimerization state of the protein and/or peptide.

As to the first of the methods, a partially unfolded or misfolded protein can still expose a specific binding site. Therefore, testing the quality of a protein and/or peptide by only testing for a specific binding site is not always a reliable method, because the partial unfolding or degradation of the protein and/or peptide is not detected.

The second of the methods, the size-related detection method is based on the concept that denaturation leads to aggregation of proteins, thereby increasing the size of the protein and/or peptide. One of several methods for detecting an increase in size of proteins is called size exclusion chromatography. Nowadays, size exclusion chromatography is widespread used as a method to analyze the contents of a protein composition. This technique is generally accepted for the testing of protein composition (http)://etd.utmem.edu/WORLD_ACCESS/vmi/reviewofanalyticmethod.htm).

Because the detection method only detects the size of a protein and/or peptide, it cannot detect misfolded proteins or proteins with increased content of cross-β structures that have not aggregated or increased in size. Therefore, both methods have disadvantages and quality control based on both the above-described methods, does not prevent undesired side effects caused by conformational changes such as, for example, cross-β structures formed upon denaturation, proteolysis, chemical modification, or unfolding of proteins, in the absence of increased molecular size. Moreover, nowadays guidelines that determine the acceptable amounts of aggregates of protein and/or peptide in solutions are based on technical limitations of the available purification methods, rather than on knowledge about expected undesired side effects of the aggregated proteins. Therefore, an improved quality control method is needed by scientists involved in development of protein and/or peptide production and formulations and for manufacturers of compositions comprising protein and/or peptide.

The present invention provides such an improved method to detect the presence of cross-β structure in a protein and/or peptide in an aqueous solution. The invention also provides methods for the removal of proteins or peptides comprising a cross-β structure conformation, thereby reducing the unwanted side effects and toxicity and increasing the specific activity per gram protein of the compositions. Therefore, the methods of the invention provide a person skilled in the art with a method of monitoring and optimizing the production methods and storing conditions of a protein and/or peptide in an aqueous solution.

In one embodiment, the present invention discloses a method for detecting a protein and/or peptide comprising a cross-β structure in an aqueous solution comprising a protein and/or peptide, the method comprising, contacting the aqueous solution comprising a protein with at least one cross-β structure-binding compound resulting in a bound protein and/or peptide comprising a cross-β structure and, detecting whether bound protein and/or peptide comprising cross-β structures are present in the aqueous solution. Binding of one or more of the cross-β structure-binding compounds to a cross-β structure is detected by means of a visualization reaction as, for example, by fluorescent staining or an enzymatic or colorimetric detection, or by any other visualization system available to a skilled person. The specification provides a number of methods for detecting the bound protein and/or peptide comprising cross-β structures and also methods for determining the amount of bound protein and/or peptide comprising cross-β structures.

Disclose are various molecules or compounds, as described in Tables 1, 2 and/or 3 of the application, alone or in combination with each other or other binding compounds, are capable of binding to a protein with a cross-β structure or a part of a protein and/or peptide essentially only comprising a cross-β structure. The term cross-β structure and cross-β structure conformation both refer to a three dimensional structure in a protein characterized by the presence of stacked or layered cross-β sheets; the terms are used interchangeably herein.

Therefore, the specification discloses a number of cross-β structure-binding compounds, with which the methods of the invention can be performed. Therefore, in another embodiment, the invention provides a method according to the invention, wherein the cross-β structure-binding compound is a compound according to Table 1, or Table 2, or Table 3 or a functional equivalent of any of the compounds. A functional equivalent of a cross-β structure-binding compound is a compound which is capable of binding to a cross-β structure.

In Tables 1, 2 and/or 3, various different compounds are described that bind to compounds with a cross-β structure. For example, Table 1 comprises among other, dyes like Thioflavin T, Thioflavin S, and Congo Red, that are used for staining amyloid molecules in histological sections or in solution. Table 2 comprises bioactive compounds binding to compounds comprising cross-β structures such as tissue-type plasminogen activator (tPA), factor XII, fibronectin, and others.

In Table 3, proteins are disclosed that are involved in the cross-β structure pathway, like for example, antibodies, heat shock proteins and receptors.

The invention also provides a protein-specific way of detecting and removing protein and/or peptide comprising cross-β structures, by combining the protein-specific binding of an antibody or a functional part thereof (i.e., a part that binds specifically to a protein), with a cross-β structure-binding compound. Therefore, the invention also provides molecular recognition units binding to compounds with cross-β structures, or single chains of antibodies. The invention further provides bi-specific recombinant binding molecules, for example, comprising the binding portion of tPA and an antibody, or the binding portion of a bioactive compound binding to proteins with cross-β structures with the binding part of an antibody.

Because of the unwanted side effects, the decrease in specific activity of a protein, and the toxicity for cells and organisms, it is preferred to know whether a protein and/or peptide comprises cross-β structures. It is disclosed in the specification how to detect a protein and/or peptide comprising a cross-β structure in a aqueous solution. The aqueous solution comprises a protein, a detergent enzyme, a food and/or a food supplement, a commercially available protein, blood and/or blood products, a cosmetic product, and/or a cell. The protein is, for example, a product comprising an enzyme for baking bread or brewing beer, or stabilizing food products. The solution also comprises, for example, enzymes used for the production of base products. The solution also comprises, for example, milk and milk products or specific protein compositions such as, for example, lubricants. The solution also comprises, for example, tissue culture fluid, for example, from recombinant production systems with prokaryotic or eukaryotic cells or from cell-free production system for recombinant production of proteins or peptides.

A person skilled in the art can now use the methods of the invention or modifications thereof to detect or deplete or detect and deplete proteins and/or peptides comprising cross-β structure from any aqueous solutions comprising a protein and/or peptide. For example, any of the compounds listed in Tables 1, 2 or 3 can be used to detect cross-β structure in, for example, aqueous solutions that are intended for use in laboratory, for example, for tissue culture, biochemistry, crystallization and so on. For example, the function of a protein can be studied before and after detection and depletion of proteins or peptides with cross-β structure. Furthermore, the present invention discloses methods to induce cross-β structure in a known protein, then select suited cross-β structure-binding compounds to the altered protein comprising a cross-β structure, and then use the binding compounds for purifying the protein product of a synthesizing and/or purification method.

Therefore, the present invention in another embodiment discloses a method for controlling a manufacturing process, and/or storage process of an aqueous solution comprising a protein, the method comprising, contacting the aqueous solution with at least one cross-β structure-binding compound resulting in a bound cross-β structure and, detecting whether bound cross-β structures are present in the aqueous solution at various stages of the manufacturing and/or storage process.

After detection of cross-β structures in a solution by a cross-β structure-binding compound, the same reaction, or optionally another reaction with a cross-β structure-binding compound is suitable for removing the cross-β structures from the solution. The bound proteins and/or peptides comprising a cross-β structure are removed by binding the cross-β structure-binding compounds to at least one other binding molecule that is bound to a solid phase, or to a third binding compound. Therefore, the invention in another embodiment discloses a method for removing a cross-β structure from an aqueous solution comprising a protein, the method comprising, contacting the aqueous solution with at least one cross-β structure-binding compound resulting in bound proteins and/or peptides comprising a cross-β structure and, allowing binding of the proteins and/or peptides comprising a cross-β structure to the cross-β structure-binding compound and, separating the bound proteins and/or peptides comprising a cross-β structure from the aqueous solution.

It is disclosed herein that the compounds of Tables 1, 2 and/or 3 of the application are suitable cross-β structure-binding compounds. Therefore, the present invention discloses a method according to the invention, wherein the cross-β structure-binding compound is a compound according to Table 1, or Table 2, or Table 3 or a functional equivalent of any of the compounds.

For efficient removal of bound proteins and/or peptides comprising a cross-β structure, a cross-β structure-binding compound is attached to another binding compound or to a solid phase by chemical or physical methods.

As a solid phase, many materials are suitable for binding a cross-β structure-binding compound, such as for example, glass, silica, polystyrene, polyethylene, nylon, vinyl, sepharose beads, beads containing iron or other metals and so on. In one embodiment of the invention, the solid phase has the physical form of beads. In another embodiment, the solid phase has the shape of a tube or a plate or a well in, for instance an ELISA plate, or a dipstick. Numerous binding techniques are available for coupling the cross-β structure-binding compounds to the solid phase, like for example, Cyanogen Bromide (CnBr), NHS, Aldehyde. epoxy, Azlactone, biotin/streptavidin, and many others. The amount of bound protein and/or peptide comprising cross-β structures is measured, for example, by staining the cross-β structures and is a measure for the quality of the proteins and/or peptides in the solution.

It generally depends on the attachment method that is selected how and when the cross-β structure-binding compound is attached to another molecule or compound. For example, a preferred binding of the compound of Table 1 to another compound occurs before binding a protein and/or peptide comprising a cross-β structure, or more preferred during the process of binding of a cross-β structure, or most preferred after binding of a protein and/or peptide comprising a cross-β structure. Therefore, the present invention discloses a method according to the invention, wherein the cross-β structure-binding compound is bound to a second compound before, during or after the binding of the cross-β structure-binding compound to a protein and/or peptide comprising a cross-β structure.

As described above, it depends on the attachment method and on the type of solid phase how and when the cross-β structure-binding compound and/or its second binding compound is attached to a solid phase. In one embodiment, the compound of Table 1 is attached to a solid phase, and in another embodiment of the invention, the compound of Tables 1, 2 or 3 or an equivalent thereof is first attached to a second binding compound, which in its turn is attached to a solid phase. Therefore, the present invention discloses a method according to the invention, wherein the second compound is bound to a solid face. For example, the second compound comprises an antibody directed against part of a compound of Tables 1, 2, or 3, or comprises a chemical linker that is capable of binding a compound of Tables 1, 2, or 3. Although in many cases it will be enough to contact a protein and/or peptide comprising a cross-β structure with a cross-β structure-binding compound, or the complex with a second binding compound, it of course also within the scope of the present invention that the second binding compound is also capable of binding to a third binding compound or even to a fourth or fifth and so on. Therefore, the present invention in another embodiment discloses a method of the invention, wherein the cross-β structure-binding compound, bound to a second compound is further bound to a third or fourth or further binding compound before, during or after the binding of the cross-β structure-binding compound to a protein and/or peptide comprising a cross-β structure. In a preferred embodiment, a second, third, or fourth, or further binding compound, is bound to a solid phase. Therefore, the present invention also discloses a method, wherein the third or fourth compound is bound to a solid phase. In another embodiment of the invention, the continued binding of more binding molecules induces the formation of aggregates, for example, by agglutination, that do not need a further solid phase to be separated from the aqueous solution.

The methods of the invention are useful for controlling the different stages of a manufacturing process of a protein and/or peptide. In general, the specification of a process for manufacturing a composition comprising a protein and/or peptide is described in a handbook according to good manufacturing practice (GMP) and good laboratory practice (GLP). GLP and GMP quality control is a valuable tool for manufacturers of protein compositions and for manufacturers of constituents comprising a protein and/or peptide and it helps and enables them to produce products of a steady quality and to increase the quality by monitoring the manufacturing and storage process. The present invention discloses methods that help manufacturers to detect compounds with cross-β structures in the product. A qualitative difference is thus made between products with cross-β structures or products without cross-β structures, or with low levels of cross-β structure. By monitoring the processes with methods of the invention, manufacturers are capable of omitting processes or chemicals or physical conditions or circumstances or treatments that induce the formation of cross-β structures, and it enables them to select processes or chemicals or circumstances that do not induce cross-β structures and/or raise the level of cross-β structures in a solution comprising a protein.

The present invention also discloses a method for decreasing and/or preventing undesired side effects of an aqueous solution comprising a protein and/or increasing the specific activity per gram protein of an aqueous solution, the method comprising detecting and removing any unfolded protein or peptide and/or aggregated protein or peptide and/or multimerized protein or peptide comprising a cross-β structure from the aqueous solution according to any method of the invention.

In one preferred embodiment, the present invention discloses a method for detecting and/or measuring a cross-β structure-inducing ability of a solid surface, by contacting the surface with a protein and detecting denatured protein by subsequently contacting the surface with a cross-β structure-binding compound. With the method of the invention, a person skilled in the art is capable of selecting materials for a container for storing protein. The same procedure is suitable for selecting a reaction vessel, a production vessel, a storage vessel and/or a tube connecting the vessels. The above-described method is also suitable for detecting and/or measuring a cross-β structure-inducing ability of a molecule, for example, of a salt, or a dye, or an enzyme, or a chemical compound such as, for example, alcohol or formaldehyde or glucose. Therefore, the present invention discloses in another embodiment a method for detecting and/or measuring a cross-β structure-inducing ability of a substance, by contacting the substance with a protein and detecting denatured protein by subsequently contacting the molecule and/or the protein with a cross-β structure-binding compound. Substances that have the ability to induce a cross-β structure are then removed or avoided in the production, purification and storage of a protein. Therefore, the present invention enables a person skilled in the art to avoid the use of substances as a part of the aqueous solution or as a part of a wall of a container for production, purification, or storage of the protein and/or peptide. In another embodiment, the invention teaches the person skilled in the art to avoid substances inducing cross-β structure in the preparation of a solution comprising a protein and/or peptide. Therefore, the present invention provides a method for selecting substances for production and/or dilution, and/or preservation of a composition comprising a protein and/or peptide.

In yet another embodiment, the present invention discloses a method for detecting and/or measuring a cross-β structure-inducing ability of a physical condition such as for example, pH, pressure, temperature, salt concentration and/or protein concentration. A recombinant protein and/or peptide is subjected to various physical conditions and the increase or induction of the amount of cross-β structures is measured by contacting the protein and/or peptide with a cross-β structure-binding compound according to a method of the invention. Binding of a protein and/or peptide comprising a cross-β structure with a cross-β structure-binding compound is detected using the methods of the invention. The above-described method is a valuable tool for detecting cross-β structure-inducing circumstances during production, purification, and storage. Therefore, the present invention discloses a process to improve production, purification and storage of product comprising a protein and/or peptide.

Because the present invention discloses how to detect cross-β structures in an aqueous solution comprising a protein, a skilled person is able to select circumstances that prevent or decrease the induction of cross-β structures during the synthesis or production or purification of a protein and/or peptide.

A protein and/or peptide, which is produced, processed or purified according to any one of the methods of the present invention, comprises less compounds with cross-β structures and is, therefore, less toxic, thrombogenic, immunogenic, inflammatory or harmful for a mammal including a human after administration of the protein and/or peptide. Furthermore, because of the decreased presence of protein and/or peptide comprising cross-β structures, the purity and the specific activity of a protein is preferably higher per gram protein present in the protein, and therefore, less protein is needed to achieve the same pharmacological effect. A protein and/or peptide that is purified by any of the methods of the invention is, therefore, of higher quality, and exerts less side effects than a protein and/or peptide that is not purified. The difference between a protein and/or peptide according to the invention and another protein and/or peptide is in the lower amount of protein and/or peptide comprising cross-β structures that is detectable in the protein and/or peptide according to the invention.

Therefore, the present invention in another embodiment provides an aqueous solution comprising a protein and/or peptide, obtainable by a method according to the invention.

In another embodiment, the specification provides a kit of parts, comprising, for example, one or more cross-β structure-binding compounds as depicted in Tables 1, or 2, or possibly 3, and optionally one or more compounds binding the cross-β structure-binding compound, and a means for detecting bound protein and/or peptide comprising a cross-β structure as described elsewhere in this specification, thereby making the kit suitable for carrying out a method according to the invention such as, for example, detecting protein and/or peptide comprising a cross-β structures, and or removing protein and/or peptide comprising a cross-β structures from a protein solution. Therefore, the present invention provides a kit for carrying out a method according to the invention, comprising all necessary means for binding a protein or peptide comprising a cross-β structure to a cross-β structure-binding compound, and/or removing a protein or peptide comprising a cross-β structure from an aqueous solution comprising a protein and/or peptide.

The presence of bound proteins or peptides with cross-β structures is in another embodiment detected by an enzymatic assay. As an example of an enzymatic assay the specification provides tPA+plasminogen+plasmin substrate S-2251 (Chromogenix Spa, Milan, Italy) in a suitable buffer. Preferably the buffer is HBS (10 mM HEPES, 4 mM KCl, 137 mM NaCl, pH 7.3). Standard curve is made with a control with a cross-β structure. Titration curves are made with a sample before and after a treatment/exposure to a putatively denaturing condition. Alternatively the detection of bound cross-β structures is achieved by a test wherein factor XII with activated factor XII substrate S-2222 or S-2302 is present in a suitable buffer. Preferably, the buffer is 50 mM, 1 mM EDTA, 0.001% v/v Triton-X100. Standard curves are made with known cross-β structure rich activators of factor XII; preferably dextran sulphate 500,000 k (DXS500k) with a protein; preferably the protein is endostatin or albumin; preferably with glycated hemoglobin, Aβ, amyloid fibrin peptide NH₂-148KRLEVDIDIGIRS160-COOH with K157G mutation (SEQ ID NO:_). In yet another embodiment, the presence of bound cross-β structures is detected by a test comprising factor XII with prekallikrein and high molecular weight kininogen and either substrate Chromozym-PK for kallikrein or a substrate for activated factor XII in a suitable buffer; preferably HBS. Standard curves are made with known cross-β structure rich activators of factor XII; preferably DXS500k or kaolin with a protein; preferably the protein is endostatin or albumin; preferably with glycated hemoglobin, Aβ, amyloid fibrin peptide NH₂-148KRLEVDIDIGIRS160-COOH with K157G mutation.

The specification provides in one embodiment of a kit, for example, a filter-like element, the element capable of binding protein and/or peptide comprising cross-β structures or binding cross-β structure-binding compounds. The filter is used to pass a solution comprising a protein and/or peptide through it. In another embodiment, the filter is used in the production or packaging of a protein and/or peptide. In another embodiment, the kit of the specification provides an ELISA plate, or a dipstick for detecting protein and/or peptide comprising cross-β structures in a composition comprising a protein and/or peptide or a filtration device for removing protein and/or peptide comprising cross-β structures from a solution comprising a protein, peptide, or polypeptide.

After removal of the protein and/or peptide comprising cross-β structures from a composition comprising a protein and/or peptide, the resulting composition is tested again to control whether the amount of protein and/or peptide comprising cross-β structures in the composition has actually decreased.

The cross-β structure-binding compounds of the invention are also suitable for detecting a cell with cross-β structures on the surface. A cell with cross-β structures on the surface is, for example, a bacterial cell, or a yeast cell or a eukaryotic cell. In biotechnological protein production systems, use is made of bacterial cells or yeast cells or eukaryotic cells to produce protein. Selection of those cell types that have less cross-β structures on the surface than other cells is advantageous for a production system, because induction of cross-β structures in the produced protein is less. Therefore, in another embodiment, the present invention also provides a method for detecting a cell comprising a cross-β structure on its surface in a collection of cells, the method comprising contacting the cell with a cross-β structure-binding molecule, and measuring binding of the molecule to the cell.

In a preferred embodiment, a collection of cells is made better suited for production of protein by removing cells with cross-β structures on the surface. Removing is achieved using the cross-β structure-binding compounds of the invention. Therefore, the present invention in another embodiment discloses a method for removing a cell comprising a cross-β structure on its surface from a collection of cells, the method comprising contacting the cell with a cross-β structure-binding molecule, and binding the molecule to a solid surface.

### DESCRIPTION OF THE FIGURES

FIG. 1: Binding of polypeptides with cross-β structures to tPA, sRAGE and fibronectin type I domains, studied with Biacore surface plasmon resonance. Panel A: tPA activation assay showing that 10 minutes centrifugation at 16,000*g of Hb-AGE and amyloid γ-globulins hardly influences the tPA activating properties of the supernatant when compared to uncentrifuged amyloid stocks. Also protein therapeutic endostatin is tested for tPA activating properties. Concentrations of potential activators were 100 μg ml⁻¹. Panel B: Binding of 32 μg ml⁻¹ Hb-AGE to tPA and sRAGE in a Biacore surface plasmon resnonance experiment. Panel C: On the same chip relatively strong binding of 62.5 μg ml⁻¹ to tPA and sRAGE is observed. Panel D: More BSA-AGE, injected at 3.9 μg ml⁻¹, binds to tPA than to sRAGE. Panel E: By testing a concentration series of Hb-AGE for binding to a Biacore CM5 chip with immobilized Fn F4-5, it is deduced that half maximum binding is obtained with 8 nM Hb-AGE (indicated with the arrow). Panel F: As a control, 25 nM native Hb was tested for binding to a Biacore chip with immobilized Fn F4-5, HGFA F and tPA F. Panel G: By testing a concentration series of endostatin it is revealed that half maximum binding to Fn F4-5 is obtained with 800 nM endostatin (arrow). Panel H: Half maximum binding of recombinant β2GPI to immobilized Fn F4-5 is obtained with 165 nM β2GPI (arrow).

FIG. 2: Presence of amyloid cross-β structures in protein solutions. Panels A-D: With protein solutions stored at the recommended temperature of 4° C., influence on Congo red- (Panel A) and ThT fluorescence (Panel B) was established as well as the ability to activate tPA (Panel C) and factor XII (Panel D), as determined with chromogenic assays which record Pls and kallikrein activity, that is established upon activation of Plg by tPA and prekallikrein by factor XII, respectively. Gelatin, Cealb and FVIII clearly enhance Congo red fluorescence. Cealb, GH and FVIII enhance ThT fluorescence. GH and insulin potentiate Plm activity. Amyloid γ-globulins at 100 μg ml⁻¹ was used as a positive control. Zinc-insulin and insulin activate factor XII. Kaolin at 150 μg ml⁻¹ was used as a positive control. Panel E: Both modified gelatin stored at 4° C. and at 37° C. show enhanced Congo red fluorescence comparable to the positive control, 25 μg ml⁻¹ Aβ. Panel F: Only modified gelatin that was stored at 37° C., and not gelatin stored at 4° C., exhibits factor XII stimulatory activity, as measured in a chromogenic kallikrein activity assay. The positive control for factor XII mediated prekallikrein activation was 150 μg ml⁻¹ kaolin. Panel G: tPA ELISA showing the binding of tPA to immobilized zinc-insulin, an antibody, FVIII and albumin. Positive control in the ELISA was Hb-AGE, that is not shown for clarity. Panel H: tPA ELISA showing the binding of tPA to immobilized Cealb and GH. K_{D}s are 23 nM for Cealb and 72 nM for GH. Panel I: TEM image of modified gelatin showing various relatively condense aggregates. The scalebar is 1 μm. Panel J: TEM image of GH showing a linear, a branched and a condense particle all apparently composed of spherical particles. The scale bar is 100 nm. Panel K: TEM image of zinc-insulin showing the appearance of insulin as thin unbranched fibrils with varying length. The scale bar represents 100 nm. Panel L: TEM image of Cealb stored at 4° C. Scale bar: 100 nm. Panel M: TEM image of insulin, stored at 4° C. Scale bare: 100 nm. Panel N: Influence of storage temperature on ThT fluorescence enhancement by Reopro. Panel O: tPA activating properties are largely dependent on the storage temperature of Reopro, as assessed in a tPA activation assay. Panel P: TEM image of ReoPro, stored at 4° C. Scale bar: 1 μm.

FIG. 3: Binding of factor XII and tPA to β₂-glycoprotein I. Panel A: Chromogenic Plg-activation assay showing the stimulatory activity of recombinant β₂GPI on the tPA-mediated conversion of Plg to Pls. The positive control was amyloid fibrin peptide FP13. Panel B: In an ELISA, recombinant β₂GPI binds to immobilized tPA, whereas β₂GPI purified from plasma does not bind. The k_{D} is 2.3 μg ml⁻¹ (51 nM). Panel C: In an ELISA, factor XII binds to purified recombinant human β₂GPI, and not to β₂GPI that is purified from human plasma, when purified factor XII is immobilized onto ELISA plate wells. Recombinant β₂GPI binds with a k_{D} of 0.9 μg ml⁻¹ (20 nM) to immobilized factor XII. Panel D: Western blot incubated with anti-human factor XII antibody. The β₂GPI was purified either from fresh human plasma or from plasma that was frozen at −20° C. and subsequently thawed before purification on a β₂GPI affinity column. Eluted fractions are analyzed on Western blot after SDS-PA electrophoresis. When comparing lanes 2-3 with 4-5, it is shown that freezing-thawing of plasma results in co-purification of factor XII together with the β₂GPI. The molecular mass of factor XII is 80 kDa. Panel E: Exposure of 25 μg ml⁻¹ β₂GPI, recombinantly produced (rβ₂GPI) or purified from plasma (nβ₂GPI), to 100 μM CL vesicles or to 250 μg ml⁻¹ dextran sulphate 500,000 Da (DXS) induces an increased fluorescence of ThT, suggestive for an increase in the amount of cross-β structure in solution. Signals are corrected for background fluorescence of CL, DXS, ThT and buffer. Panel F: Binding of tPA and K2P tPA to β₂GPI immobilized on the wells of an ELISA plate, or to β₂GPI bound to immobilized CL is assessed. B₂GPI contacted to CL binds tPA to a higher extent than β2GPI contacted to the ELISA plate directly. K2P tPA does not bind to β₂GPI. TPA does not bind to immobilized CL. Panel G: Transmission electron microscopy images of 400 μg ml⁻¹ purified plasma β2GPI alone (1) or contacted with 100 μM CL (2, 3) and of 400 μg ml⁻¹ purified recombinant β2GPI (4).

FIG. 4: Amyloid-like cross-β structure in alkylated murine serum albumin and in heat-denatured ovalbumin, murine serum albumin, human glucagon and Etanercept. Panel A: Plg-activation assay with Pls activity read-out using chromogenic substrate S-2251. Activating properties of reduced and alkylated murine serum albumin (alkyl-MSA) and heat-denatured ovalbumin (dOVA) are compared with amyloid γ-globulins (positive control), buffer (negative control), and native albumin and ovalbumin (nMSA, nOVA). Panel B: Thioflavin T fluorescence assay with native and denatured MSA and OVA. Panel C: tPA activation assay for comparison of reduced and alkylated MSA and heat-denatured MSA. Panel D: ThT fluorescence assay with reduced/alkylated MSA and heat-denatured MSA. Panel E: tPA activation assay with concentration series of heat/acid denatured glucagon. Panel F: ThT fluorescence assay with native and heat/acid denatured glucagon. Panel G: Comparison of the tPA activating properties of heat-denatured Etanercept, native Etanercept and reduced/alkylated Etanercept. Panel H: ThT fluorescence of native and heat-denatured Etanercept. Panel I: TEM image of heat-denatured ovalbumin. The scale bar represents 200 nm. Panel J: TEM image of heat/acid-denatured glucagon. The scale bar represents 1 μM. Panel K: ThT fluorescence assay showing that filtration through a 0.2 μm filter of denatured OVA does not influence the fluorescence enhancing properties.

### DETAILED DESCRIPTION OF THE INVENTION

Examples of useful applications of a method according to the invention are provided above and even more examples are provided below. In general it can be said that if one wants to study or obtain a protein with a particular property, it is important to check each and every treatment on their cross-β structure inducing capabilities on the protein. If, for example, a protein is used in the food industry or as a biochemical compound in research (for example, biomedical research, or in diagnostics it is important to check the production, purification and storage conditions. If one wants to study the activity of a protein (for example, an enzyme) it is important to study all the conditions to which such a protein is subjected.

Other, non-limiting, applications of a method according to the invention are:
testing of conditions for producing, purifying and storing proteins used for growing crystals for protein crystallography purposes; some of the presently used conditions result in the formation of cross-β structure in a protein and hence hamper the growth of high-quality crystals of the protein; conditions (to be) used in crystallography are now tested for their cross-β structure inducing capability and a selection is made for conditions that do not or only slightly induce the formation of cross-β structure in a protein;
testing of chemical/biochemical/biophysical conditions used in protein purifications; independent of the source of protein (naturally expressed or recombinantly expressed) proteins are typically subjected to one or multiple purification steps to obtain high grade preparations comprising a protein and/or peptide. All treatments performed with a protein or peptide in such purifications, such as buffer composition, temperature, column material, dialysis membranes, membranes used for concentration, is checked with a method according to the invention and conditions are selected that do not or only slightly induce cross-β structure formation in the to be purified protein;
testing of conditions and/or solutions for protein refolding from an aggregated state to a native fold; independent of the source of the protein with non-native fold (naturally expressed or recombinantly expressed; for example, Escherichia coli inclusion bodies), proteins are typically subjected to exposure to one or more solutions that putatively aid the folding from a non-native fold to a native fold. The solutions are now checked with a method according to the invention for their propensity to induce the cross-β structure in proteins by testing the content of cross-β structure in the proteins after the exposure to the solutions. Solutions can now be selected that do not result in cross-β structure and thus may aid the adoption of a native fold;
selection and development of cell culture solutions or laboratory liquid equipment comprising proteins or peptides in general.

It is revealed in the specification that several physical/chemical conditions influence the fold of a protein. Exposure to CL or DXS500k, a freeze-thaw cycle, variations in protein purification protocol, heating, change in pH, the source of the protein and exposure to plastic all introduce a structural rearrangement in the protein accompanied by the formation of the amyloid-like cross-β structure fold. This new fold can be detected by, amongst others, tPA binding, tPA activation, factor XII binding and by conventional amyloid fluorescence assays.

The invention is further explained in the examples, without being limited by them.

TABLE 1
cross-β structure-binding compounds

| Congo red | Chrysamine G | Thioflavin T |
|---|---|---|
| 2-(4′-(methylamino)phenyl)-6- | Any other | Glycosaminoglycans |
| methylbenzothiaziole | amyloid-binding | |
| | dye/chemical | |
| Thioflavin S | Styryl dyes | BTA-1 |
| Poly(thiophene acetic acid) | conjugated | |
| | polyeclectrolyte | |
| | PTAA-Li | |

TABLE 2
cross-β structure-binding proteins

| | Finger domain(s) of tPA, | |
|---|---|---|
| Tissue-type plasminogen | factor XII, fibronectin, | |
| activator | HGFA | Apolipoprotein E |
| Factor XII | Plasmin(ogen) | Matrix metalloprotease-1 |
| Fibronectin | 75 kD-neurotrophin receptor | Matrix metalloprotease-2 |
| | (p75NTR) | |
| Hepatocyte growth factor | α2-macroglobulin | Matrix metalloprotease-3 |
| activator | | |
| Serum amyloid P | High molecular weight | Hybridoma antibody |
| component | kininogen | 2C11(F8A6)^{‡} |
| C1q | Cathepsin K | Hybridoma antibody |
| | | 4A6(A7)^{‡} |
| CD36 | Matrix metalloprotease 9 | Hybridoma antibody 2E2(B3)^{‡} |
| Receptor for advanced | Haem oxygenase-1 | Hybridoma antibody 7H1(C6)^{‡} |
| glycation end-products | | |
| Scavenger receptor-A | low-density lipoprotein | Hybridoma antibody |
| | receptor-related protein | 7H2(H2)^{‡} |
| | (LRP) | |
| Scavenger receptor-B | DnaK | Hybridoma antibody 7H9(B9)^{‡} |
| ER chaperone ERp57 | GroEL | Hybridoma antibody 8F2(G7)^{‡} |
| Calreticulin | VEGF165 | Hybridoma antibody 4F4^{‡} |
| Hybridoma | Hybridoma conformational | Amyloid oligomer-specific |
| conformational antibody | antibody WO2 (ref.¹⁵) | antibody (ref.⁹) |
| WO1 (ref.¹⁵) | | |
| formyl peptide | α(6)β(1)-integrin | CD47 |
| receptor-like 1 | | |
| Rabbit anti-albumin-AGE | CD40/CD40-ligand | apo A-I belonging to small |
| antibody, Aβ-purified^{a)} | | high-density lipoproteins |
| apoJ/clusterin | 10 times molar excess | |
| | PPACK, 10 mM εACA, | |
| | (100 pM-500 nM) tPA^{b)} | |

‡hybridoma antibodies developed in collaboration with the ABC-Hybridoma Facility, Utrecht University, Utrecht, the Netherlands.
^{a)}Antigen albumin-AGE and ligand Aβ were send in to Davids Biotechnologie (Regensburg, Germany); a rabbit was immunized with albumin-AGE, antibodies against a structural epitope were affinity purified using a column with immobilized Aβ.
^{b)}PPACK is Phe-Pro-Arg-chloromethylketone, εACA is ε-amino caproic acid, tPA is tissue-type plasminogen activator

TABLE 3
Proteins likely to be involved in the “Cross-β structure pathway”

| | | |
|---|---|---|
| Hybridoma antibody 4B5 | Heat shock protein 27 | Heat shock protein 40 |
| Hybridoma antibody 3H7^{‡} | Nod2 (= CARD15) | Heat shock protein 70 |
| FEEL-1 | Pentraxin-3 | HDT1 |
| LOX-1 | Serum amyloid A | GroES |
| | proteins | |
| MD2 | Stabilin-1 | Heat shock protein 90 |
| FEEL-2 | Stabilin-2 | CD36 and LIMPII |
| | | analogous-I (CLA-1) |
| macrophage receptor with collagenous | LPS-binding | CD14 |
| structure (MARCO) | protein | |
| C reactive protein | CD45 | orosomucoid |
| Integrins | alpha-1 antitrypsin | apo A-IV-TTR |
| | | complex |
| Albumin | Alpha-1 acid | β2-glycoprotein I |
| | glycoprotein | |
| Lysozyme | Lactoferrin | megalin |
| Tamm-Horsfall protein | Apolipoprotein E3 | Apolipoprotein E4 |
| Toll-like receptors | Complement | CD11d/CD18 (subunit |
| | receptor | aD) |
| | CD11b/CD18 | |
| | (Mac-1, CR3) | |
| CD11b2 | CD11a/CD18 | CD11c/CD18 (CR4, |
| | (LFA-1, subunit | subunit aX) |
| | aL) | |
| Von Willebrand factor | | |

‡hybridoma antibodies developed in collaboration with the ABC-Hybridoma Facility, Utrecht University, Utrecht, The Netherlands.

### EXAMPLES

Materials and Methods

Preparation of Cross-β Structure Rich Compounds

For preparation of advanced glycation end-product (AGE)-modified bovine serum albumin, 100 mg ml⁻¹ of albumin was incubated with phosphate buffered saline pH 7.3 (PBS) containing 1 M of glucose-6-phosphate (g6p) and 0.05% m/v NaN₃, at 37° C. in the dark. Glycation was prolonged up to 23 weeks.⁵ To prepare glycated hemoglobin (Hb-AGE), human hemoglobin (Hb, Sigma-Aldrich, H7379) at 5 mg ml⁻¹ was incubated for 32 weeks at 37° C. with PBS containing 1 M of g6p and 0.05% m/v of NaN₃. In control solutions, g6p was omitted. After incubations, solutions were extensively dialyzed against distilled H₂O and, subsequently, stored at 4° C. Protein concentrations were determined with advanced protein-assay reagent ADV01 (Cytoskeleton, Denver, Colo., US). Glycation and formation of AGE was confirmed by measuring intrinsic fluorescent signals from AGE; excitation wavelength 380 nm, emission wavelength 435 nm. In addition, binding of AGE-specific antibodies was determined. Presence of cross-β structure or cross-β structure conformation in albumin-AGE was confirmed by enhancement of Congo red fluorescence, enhancement of Thioflavin T (ThT) fluorescence, the presence of β-sheet secondary structure, as observed with circular dichroism spectropolarimetry (CD) analyzes, and by X-ray fiber diffraction experiments.⁵ Presence of cross-β structures in Hb-AGE was confirmed by tPA binding, CD analyses, transmission electron microscopy (TEM) imaging of fibrillar structures and by Congo red fluorescence measurements. Amyloid preparations of human γ-globulins were made as follows. Lyophilized γ-globulins (G4386, Sigma-Aldrich) were dissolved in a 1(:)1 volume ratio of 1,1,1,3,3,3-hexafluoro-2-propanol and trifluoroacetic acid and subsequently dried under an air stream. Dried 7-globulins were dissolved in H₂O to a final concentration of 1 mg ml⁻¹ and kept at room temperature for at least three days, or kept at 37° C. for three days and subsequently at −20° C. Aliquots were stored at −20° C. and analyzed for the presence of cross-β structures. Fluorescence of Congo red and ThT was assessed. In addition tPA binding was analyzed in an ELISA and tPA activating properties in a chromogenic plasminogen (Plg) activation assay. In addition, the macroscopic appearance of denatured γ-globulins was analyzed with TEM imaging.

Human amyloid-β (Aβ) (1-40) Dutch type (DAEFRHDSGYEVHHQKLVFFAQDVGSNKGAIIGLMVGGVV) (SEQ ID NO:_), was disaggregated in a 1:1 (v/v) mixture of 1,1,1,3,3,3-hexafluoro-2-isopropyl alcohol and trifluoroacetic acid, air-dried and dissolved in H₂O (10 mg ml⁻¹). After three days at 37° C., the peptide was kept at room temperature for two weeks, before storage at 4° C. Aβ solutions were tested for the presence of amyloid conformation by ThT or Congo red fluorescence and by TEM imaging. Negative control for cross-β structure detection assays was non-amyloid fragment FP10 of human fibrin α-chain(148-157) (KRLEVDIDIK) (SEQ ID NO:_).^{16, 17} FP10 was dissolved at a concentration of 1 mg ml⁻¹ in H₂O and stored at 4° C. This solution was used as a negative control for ThT fluorescence assays.

Cloning and Expression of Recombinant Fibronectin Type I Domains

F4-5 domains and the F domain of tPA with a carboxy-terminal His₆-tag were also expressed in Saccharomyces cerevisiae. The cDNA constructs were prepared following standard procedures,¹⁸ by the Biotechnology Application Center (BAC-Vlaardingen/Naarden, The Netherlands). Domain boundaries of Fn F4-5 and tPA F were taken from the human Fn and human tPA entries in the Swiss-Prot database (P02751 for Fn, P00750 for tPA) and comprised amino-acids NH₂-1182-V276-COOH of Fn F4-5 and NH₂-G33-S85-COOH of tPA. Affinity purification of the expressed proteins was performed using His₆-tag-Ni²⁺ interaction and a desalting step. Constructs were stored at −20° C. in PBS pH 7.0. The molecular size of the constructs was checked on a Coomassie brilliant blue-stained SDS-PAGE gel.

Totally Chemical Synthesis of Fibronectin Type I Domains

Totally chemical synthesis of the F domains of hepatocyte growth factor activator (HGFA, SwissProt entry Q04756) and tPA (SwissProt entry P00750) was performed in the laboratory of Dr. T. M. Hackeng (Academic Hospital Maastricht, The Netherlands), according to standard procedures.¹⁹ Both domains were synthesized as two separate peptides that were subsequently ligated using native chemical ligation. The tPA F domain was completed with a carboxy-terminal acetylated lysine residue or biotinylated lysine residue. The HGFA F domain was supplied with an acetylated lysine residue. Products were analyzed on a reversed phase HPLC column and with mass spectrometry.

Cloning, Expression and Purification of the Soluble Extracellular Domains of Receptor for Advanced Glycation End-Products

The soluble extracellular part, of the receptor for AGE (sRAGE) was cloned, expressed and purified as follows (Q.-H. Zeng, Prof. P. Gros, Dept. of Crystal- & Structural Chemistry, Bijvoet Center for Biomolecular Research, Utrecht University, Utrecht, The Netherlands). Human cDNA of RAGE was purchased from RZPD (clone IRALp962E1737Q2, RZPD, Berlin, Germany). For PCRs, the gagatctGCTCAAAACATCACAGCCCGG (SEQ ID NO:_) forward primer was used comprising a BglII site, and the gcggccgcCTCGCCTGGTTCGATGATGC (SEQ ID NO:_) reverse primer with a NotI site. The soluble extracellular part of RAGE comprises three domains spanning amino-acid residues 23-325. The PCR product was cloned into a pTT3 vector, containing an amino-terminal His-tag and a thrombin cleavage site. The sRAGE was expressed in 293E hamster embryonic kidney cells at the ABC-protein expression facility (Utrecht University, Utrecht, The Netherlands). Concentrated cell culture medium was applied to a Hi-trap Chelating HP Ni²⁺-NTA column (Amersham Biosciences Europe, Roosendaal, The Netherlands). The running buffer was 25 mM Tris-HCl, 500 mM NaCl, pH 8.0. The protein was eluted by using a step gradient of 0 to 500 mM imidazole. Purity of the His-sRAGE was depicted from Coomassie stained SDS-PAGE gels. After concentration, the buffer was exchanged to 20 mM Tris-HCl, 200 mM NaCl, 100 μM phenylmethylsulfonyl fluoride (PMSF), pH 8.0. Various stocks at 1, 5 and 20 mg ml⁻¹ were first kept at 4° C. for several weeks and then stored at −20° C. In this way, the PMSF will be sufficiently inactivated at 4° C.

Plasminogen-Activation Assay and Factor XII Activation Assay

Plasmin (Pls) activity was assayed as described.¹⁶ Peptides and proteins that were tested for their stimulatory ability were regularly used at 100 μg ml⁻¹. The tPA and plasminogen (Plg) concentrations were 200 pM and 1.1 μM, respectively, unless stated differently. Chromogenic substrate S-2251 (Chromogenix, Instrumentation Laboratory SpA, Milano, Italy) was used to measure Pls activity. Conversion of zymogen factor XII (#233490, Calbiochem, EMD Biosciences, Inc., San Diego, Calif.) to proteolytically active factor XII (factor XIIa) was assayed by measurement of the conversion of chromogenic substrate Chromozym-PK (Roche Diagnostics, Almere, The Netherlands) by kallikrein. Chromozym-PK was used at a concentration of 0.3 mM. Factor XII, human plasma prekallikrein (#529583, Calbiochem) and human plasma cofactor high-molecular weight kininogen (#422686, Calbiochem) were used at concentrations of 1 μg ml⁻¹. The assay buffer contained HBS (10 mM HEPES, 4 mM KCl, 137 mM NaCl, 5 μM ZnCl₂, 0.1% m/v albumin (A7906, Sigma, St. Louis, Mo., USA), pH 7.2). Assays were performed using microtiter plates (Costar, Cambridge, Mass., USA). Peptides and proteins were tested for their ability to activate factor XII. 150 μg ml⁻¹ kaolin, an established activator of factor XII was used as positive control and solvent (H₂O) as negative control. The conversion of Chromozym-PK was recorded kinetically at 37° C. for at least 60 minutes. Assays were done in duplicate. In control wells factor XII was omitted from the assay solutions and no conversion of Chromozym-PK was detected. In some assays albumin was omitted from the reaction mixture. Alternatively, chromogenic substrate S-2222 (Chromogenix) was used to follow the activity of factor XII itself. With S-2222, activation of factor XII in plasma was measured, using 60% v/v plasma, diluted with substrate and H₂O with or without potential cofactor. Furthermore, auto-activation of factor XII was measured by incubating 53 μg ml⁻¹ purified factor XII in 50 mM Tris-HCl buffer pH 7.5 with 1 mM EDTA and 0.001% v/v Triton-X100, with S-2222 and H₂O with or without potential cofactor.

Surface Plasmon Resonance Studies

Binding of cross-β structure containing peptides/proteins was studied using surface plasmon resonance technology with a Biacore 2000 apparatus (Biacore AB, Uppsala, Sweden). A standardized amine coupling procedure was used to couple proteins with F domains to a CM5 chip (Biacore AB, Uppsala, Sweden). First, the dextran surface of the chips was activated by a 35 μl injection with a 1:1 mixture of 0.1 M N-hydroxysuccinimide (NHS) and 0.4 M N-ethyl-N′-(dimethylaminopropyl)carbodiimide (EDC) at a flow rate of 5 μl minute⁻¹. Then, the proteins were covalently coupled to the activated dextran surface. Remaining activated groups in each of the four flow channels were blocked by injection of 35 μl of 1 M ethanolamine hydrochloride pH 8.5. EDC, NHS and ethanolamine hydrochloride were obtained from Biacore. On one chip, on channels 1 to 4, buffer (reference channel), the soluble extracellular part of receptor for advanced glycation end-products (sRAGE), tPA and K2P-tPA were immobilized. The immobilization buffer for the reference channel, channel 2 (sRAGE), channel 3 (tPA) and channel 4 (K2P-tPA) was 10 mM acetate pH 3.75. In channel 2, 2000 response units (RU) sRAGE was immobilized, 2700 RU and 2400 RU tPA and K2P-tPA are immobilized, respectively. The flow rate was 10 μl minute⁻¹, the injection time was 120 seconds. The running buffer during immobilization was 10 mM HEPES pH 7.4, 140 mM NaCl. Buffers were filtrated on a 0.22 μm filter (white GSWP, 47 mm, Millipore) and degassed at room temperature. For subsequent binding experiments, the running buffer was 10 mM HEPES pH 7.4, 140 mM NaCl, 1.5 mM CaCl₂, 10 mM FACA, 0.005% Tween-20. Binding of albumin-AGE was determined with a solution of 3.9 μg ml⁻¹ albumin-AGE in running buffer. Albumin-AGE was filtered on a Millex-GV 0.22 μm filter unit (Millipore). Binding of filtered Hb-AGE was tested at 32 μg ml⁻¹. Binding of amyloid γ-globulins were tested at 62.5 μg ml⁻¹. After each injection of protein, the chip was regenerated with 0.1 M H₃PO₄ pH 1.0. After injections with albumin-AGE and Hb-AGE this regeneration step was successful and sufficient, after injection with amyloid γ-globulins, the bound protein could not be released, not even after injection with more harsh regeneration buffers (HCl, NaOH). Binding of Hb-AGE was also tested after centrifugation for 10 minutes at 16,000*g alternative to filtration. tPA activation before and after filtration was assessed with a Plg-activation assay. Also amyloid γ-globulins and amyloid endostatin (EntreMed, Inc., Rockville, Md., USA) were tested before and after centrifugation.

On a second chip, buffer, chemically synthesized HGFA F domain, chemically synthesized tPA F domain and Fn F4-5-His6, expressed in S. cerevisiae, were immobilized. HGFA F was immobilized in 10 mM acetate buffer pH 4.0, 190 RU. tPA F was immobilized in 5 mM maleate pH 5.5, 395 RU, Fn F4-5 in 5 mM maleate pH 6.0, 1080 RU. Now, the running buffer was 10 mM HEPES pH 7.4, 140 mM NaCl, 1.5 mM CaCl₂, 10 mM FACA, 0.05% Tween-20. Regeneration buffer was running buffer supplemented with 1 M NaCl. Binding was tested with endostatin at 0-800 nM, Hb-AGE at 0-25 nM, recombinant β2-glycoprotein I (β2GPI) at 0-300 nM and 25 nM native Hb. For the Fn F4-5 channel, the maximum binding expressed in RU was plotted against the concentrations.

For both chips, channel 1 was used for reference purposes. The signal obtained with this channel was subtracted from the signals obtained with the channels with immobilized proteins.

Thioflavin T Fluorescence

Fluorescence of ThT—protein/peptide adducts was measured as follows. Solutions of 25 μg ml⁻¹ of protein or peptide preparations were prepared in 50 mM glycine buffer pH 9.0 with 25 μM ThT. Fluorescence was measured at 485 nm upon excitation at 435 nm. Background signals from buffer, buffer with ThT and protein/peptide solution without ThT were subtracted from corresponding measurements with protein solution incubated with ThT. Regularly, fluorescence of Aβ was used as a positive control, and fluorescence of FP10, a non-amyloid fibrin fragment,¹⁶ was used as a negative control. Fluorescence was measured in triplicate on a Hitachi F-4500 fluorescence spectrophotometer (Ltd., Tokyo, Japan).

Congo Red Fluorescence

Solutions of 25 μg ml⁻¹ protein/peptide were incubated with 25 μM Congo red in PBS and fluorescence was measured at 590 nm upon excitation at 550 nm. Background signals from buffer, buffer with Congo red and protein/peptide solution without Congo red were subtracted from corresponding measurements with protein solution incubated with Congo red. Fluorescence was measured in triplicate on a Hitachi F-4500 fluorescence spectrophotometer (Ltd., Tokyo, Japan).

Transmission Electron Microscopy Imaging

For TEM analysis of protein en peptide solutions grids were prepared according to standard procedures. Samples were applied to 100-mesh copper grids with carbon coated Formvar (Merck, Germany), and subsequently washed with PBS and H₂O. Grids were applied to droplets of 2% (m/v) methylcellulose with 0.4% (m/v) uranylacetate pH 4. After a 2-minute incubation, grids were dried on a filter. Micrographs were recorded at 80 kV, at suitable magnifications on a JEM-1200EX electron microscope (JEOL, Japan).

Structural Analysis of Formulated Protein Therapeutics

Formulated protein therapeutics were obtained from the local hospital pharmacy and were used as supplied by the manufacturers. The following protein therapeutics were purchased: 1) human growth hormone (GH) (Genotropin, batch 52344B51, 5 mg ml⁻¹ KabiQuick, Pharmacia B. V., Woerden, The Netherlands), 2) recombinant human Zn²⁺-chelated insulin (Monotard, batch NS61694, 100 IE ml⁻¹, Novo Nordisk, Bagsvaerd, Denmark), 3) human albumin (Cealb, batch NS61694, 200 mg ml⁻¹, Sanquin-CLB, Amsterdam, The Netherlands), 4) human-modified gelatin (Gelofusine, batch 030606H4, 40 mg ml⁻¹, Braun Medical BV, Oss, The Netherlands), 5) rapid acting human insulin analogue (NovoRapid Flexpen, batch PH70008, 10 U ml⁻¹, Novo Nordisk), 6) blood cell growth factor filgrastim (Neupogen Singleject, batch N0693AD, 960 μg ml⁻¹, Amgen Europe, Breda, The Netherlands), 7) human-murine chimeric monoclonal antibody (Remicade-infliximab, batch 03D06H120A, 10 mg ml⁻¹, Centocor, Leiden, The Netherlands), 8) abciximab, an inhibitor of blood platelet aggregation (ReoPro, 2 mg ml⁻¹ , Centocor, Leiden, The Netherlands) and 9) human coagulation factor VIII (FVIII) isolated from healthy volunteers (Aafact, lot 02L046250A, 3.6 mg ml⁻¹, Sanquin-CLB, Amsterdam, The Netherlands). Lyophilized therapeutics were dissolved according to the manufacturers recommendations. GH, zinc-insulin, Cealb and gelatin were stored at −20, 4, room temperature, 37 and 65° C. Other protein therapeutics were only kept at 4° C., and assayed for the presence of cross-β structure at shown time points. Enhancement in fluorescence of ThT and Congo red was measured with all formulated protein therapeutics. For this purpose, proteins were diluted to the indicated concentrations. In addition, tPA binding to the protein therapeutics was analyzed by ELISA and activation of tPA was tested using the Plg-activation assay. Zinc-insulin was diluted tenfold in the activation assay, GH was diluted to a final concentration of 500 μg ml⁻¹. Activation of factor XII and prekallikrein by the therapeutics was tested in the chromogenic factor XII assay (see above). For tPA ELISAs, 5 μg ml⁻¹ of the protein therapeutics were coated onto Greiner high-binding Microlon plates (#655092, Greiner Bio-One, Alphen a/d Rijn, The Netherlands). After coating, plates were blocked with Blocking Reagent (Roche Diagnostics, Almere, The Netherlands). A concentration series of tPA or K2P-tPA in PBS with 0.1% v/v Tween-20 and 10 mM E-amino caproic acid was applied and the plates were incubated for one hour at room temperature with constant swirling. Binding of tPA was assessed with monoclonal antibody 374b that binds to the protease domain of both tPA and K2P-tPA (American Diagnostica, Tebu-Bio, The Netherlands), peroxidase-conjugated rabbit anti-mouse immunoglobulins (RAMPO, P0260, DAKOCytomation, Glostrup, Denmark), and stained with 3′3′5′5′-tetramethylbezidine (TMB, catalogue number 4501103, buffer, catalogue number 4501401, Biosource Int., Camarillo, Calif., USA).

Activation of tPA by β₂-glycoprotein I, Binding of Factor XII and tPA to β₂-glycoprotein I, and ThT and TEM Analysis of P₂-glycoprotein I

Purification of β₂-glycoprotein I (β2GPI) was performed according to established methods.^{20, 21} Recombinant human β₂GPI was made using insect cells and purified as described.²⁰ Plasma derived β₂GPI as used in a factor XII ELISA, the chromogenic Plg-activation assay, was purified from fresh human plasma as described.²¹ Alternatively, β₂GPI was purified from, either fresh human plasma, or frozen plasma (−20° C.) on an anti-β₂GPI antibody affinity column.²²

Activation of tPA (Actilyse, Boehringer-Ingelheim) by β₂GPI preparations was tested in the Plg-activation assay (see above). Hundred μg ml⁻¹ plasma β₂GPI or recombinant β₂GPI were tested for their stimulatory cofactor activity in the tPA-mediated conversion of Plg to Pls, and were compared to the stimulatory activity of peptide FP13.¹⁶

Binding of purified human factor XII from plasma (Calbiochem) or of purified recombinant human tPA to β₂GPI purified from human plasma, or to recombinant human β₂GPI was tested in an ELISA. Ten μg of factor XII or tPA in PBS was coated onto wells of a Costar 2595 ELISA plate (Cambridge, Mass., USA) and incubated with concentration series of the two β₂GPI preparations. Binding of β₂GPI was assessed with monoclonal antibody 2B2.²²

Binding of factor XII to β₂GPI was also tested using immunoblotting. β₂GPI (33 μg) purified either from fresh plasma or from frozen plasma was brought onto a 7.5% SDS-PAGE gel. After blotting to a nitrocellulose membrane, the blot was incubated with 1000× diluted rabbit polyclonal anti-human factor XII antibody (#233504, Calbiochem) and after washing with 3000× diluted peroxidase-conjugated swine anti-rabbit immunoglobulins (SWARPO, #P0399, DAKOCytomation, Glostrup, Denmark).

ThT fluorescence of β₂GPI was measured as follows. Purified β₂GPI from human plasma (400 μg ml⁻¹ final concentration) was incubated with or without 100 μM cardiolipin (CL) vesicles or 250 μg ml⁻¹ of the factor XII activator dextran sulphate 500 k (DXS500k, Pharmacia, Uppsala, Sweden), in 25 mM Tris-HCl, 150 mM NaCl, pH 7.3. CL vesicles were prepared according to an established procedure. Briefly, CL was dried under a stream of nitrogen. The lipids were resuspended to a concentration of 10 mg ml⁻¹ in 25 mM Tris-HCl, pH 7.3, 150 mM NaCl by vigorous agitation, using a vortex. In the ThT fluorescence assay, fluorescence of β₂GPI in buffer, of CL or DXS500k in buffer, of buffer and ThT alone, and of β₂GPI-CL adducts and β₂GPI-DXS500k adducts, with or without ThT, was recorded as described above (section ThT fluorescence). In addition, TEM images were recorded with CL, β2GPI from human plasma, with or without CL, and with recombinant β2GPI, as described.⁵

Preparation of Amyloid-Like Ovalbumin, Human Glucagon, Etanercept and Murine Serum Albumin

To prepare structurally altered ovalbumin (OVA) with amyloid cross-β structures, purified OVA (Sigma, A-7641, lot 071k7094) was heated to 85° C. One mg ml⁻¹ OVA in 67 mM NaPᵢ buffer pH 7.0, 100 mM NaCl, was heated for two cycles in PCR cups in a PTC-200 thermal cycler (MJ Research, Inc., Waltham, Mass., USA). In each cycle, OVA was heated from 30 to 85° C. at a rate of 5° C./minute. Native OVA (nOVA) and heat-denatured OVA (dOVA) were tested in the ThT fluorescence assay and in the Plg-activation assay. In the fluorescence assay and in the Plg-activation assay, 25 and 100 μg ml⁻¹ nOVA and dOVA were tested, respectively. TEM images of nOVA and dOVA were taken to check for the presence of large aggregates.

Modified murine serum albumin (MSA) was obtained by reducing and alkylation. MSA (#126674, Calbiochem) was dissolved in 8 M urea, 100 mM Tris-HCl pH 8.2, at 10 mg ml⁻¹ final concentration. Dithiothreitol (DTT) was added to a final concentration of 10 mM. Air was replaced by N₂ and the solution was incubated for two hours at room temperature. Then, the solution was transferred to ice and iodoacetamide was added from a 1 M stock to a final concentration of 20 mM. After a 15-minute incubation on ice, reduced-alkylated MSA (alkyl-MSA) was diluted to 1 mg ml⁻¹ by adding H₂O. Alkyl-MSA was dialyzed against H₂O before use. Native MSA (nMSA) and alkyl-MSA were tested in the ThT fluorescence assay and in the Plg-activation assay. In the ThT-fluorescence assay 25 μg ml⁻¹ nMSA and alkyl-MSA were tested, and in the Plg-activation assay 100 μg ml⁻¹ was tested. The presence of aggregates or fibrils was analyzed using TEM.

Amyloid-like properties in human glucagon (Glucagen, #PW60126, Novo Nordisk, Copenhagen, Denmark) were introduced using a modified protocol based on the method described by Onoue et al.²³ Lyophilized sterile glucagon was dissolved at 1 mg ml⁻¹ in H₂O with 10 mM HCl. The solution was subsequently kept at 37° C. for 24 hours, at 4° C. for 14 days and again at 37° C. for nine days. ThT fluorescence was determined as described above, and compared with freshly dissolved glucagon. tPA-activating properties of both heat-denatured glucagon and freshly dissolved glucagon was tested at 50 μg ml⁻¹. TEM analysis was performed to assess the presence of large multimeric structures.

### Example 1

Protein Assemblies with Cross-β Structure Bind to Immobilized Fibronectin Type I Domains in a Biacore Surface Plasmon Resonance Set-Up

We used a surface plasmon resonance set-up of Biacore to test whether immobilized proteins with affinity for cross-β structure can capture amyloid-like polypeptides from solution under flow. This set up also allows testing of suitable elution buffers to disrupt the interaction. In this way insight into suitable methods to deplete proteins with cross-β structures from solutions is obtained, as well as insight into how to compete for the interaction of cross-β structure binders, which are, for example, immobilized on beads in a column, with proteins comprising cross-β structures.

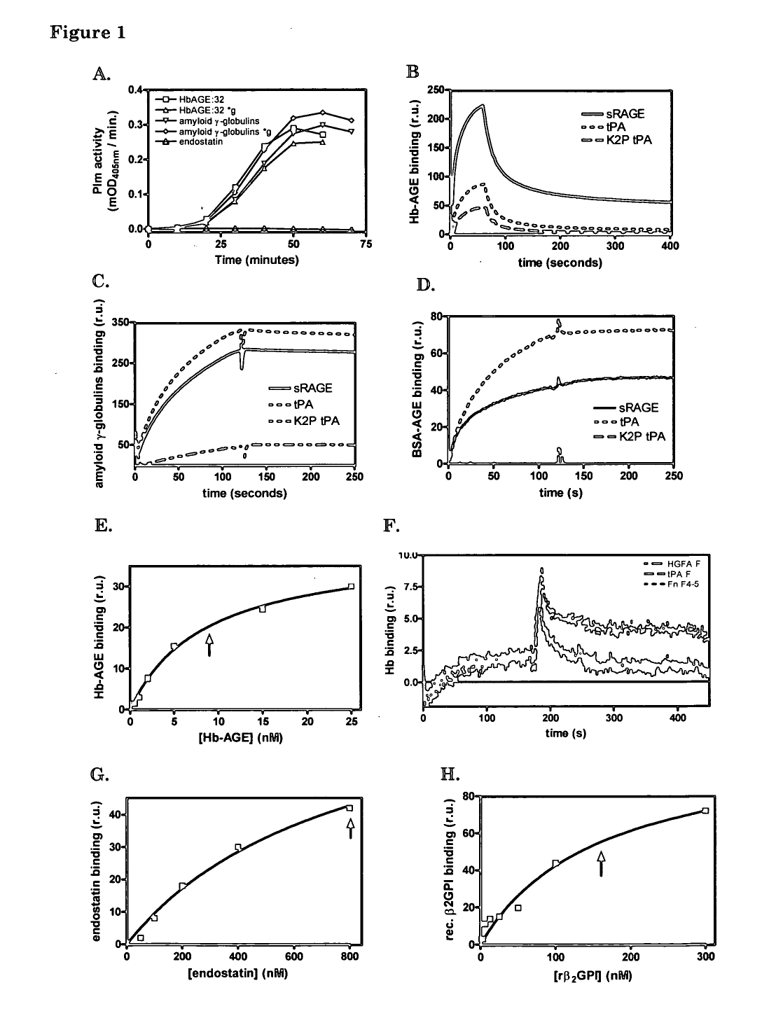

On one chip, we immobilized sRAGE, tPA and K2P-tPA. One channel was left empty for reference purposes. Protein solutions were centrifuged for 10 minutes at 16,000*g before the solutions were applied to the Biacore chip. Centrifugation had no effect on the stimulatory effect of Hb-AGE and amyloid β-globulins on tPA-mediated activation of Plg (FIG. 1, Panel A). Moreover, we filtrated all protein solutions before they were applied to the Biacore to exclude the presence of large aggregates with a density equal to buffer. For Hb-AGE similar response units were obtained after centrifugation or filtration (not shown). Subsequent experiments showed that Hb-AGE, albumin-AGE and amyloid γ-globulins bind to immobilized tPA and sRAGE (FIG. 1, Panels B-D). The interaction of tPA and sRAGE with Hb-AGE and albumin-AGE could be disrupted with 0.1 M H₃PO₄ buffer pH 1.0. Amyloid γ-globulins, however, were not removed by this buffer. After trying several more harsh regeneration buffers, the binding capacity of the chip was lost.

On a second chip, chemically synthesized HGFA F and tPA F, and Fn F4-5-His expressed in S. cerevisiae were immobilized. None of the polypeptides with cross-β structures bound to the two single F domain constructs. Hb-AGE, endostatin and recombinant β2GPI bound, however, to the Fn F4-5 doublet, whereas native Hb did hardly bind (FIG. 1, Panels E-H). Affinities of the three proteins for Fn F4-5, expressed as the concentration of ligand that results in half maximum binding, ranges from 8 nM for Hb-AGE, via 165 nM for recombinant β2GPI to up to 800 nM for endostatin. In fact, based on the absence of tPA activating properties in 100 μg ml⁻¹ endostatin (FIG. 1, Panel A), we did not expect any binding at all. Putatively, the surface plasmon resonance is more sensitive for the cross-β structure under the conditions used. We observed that when a stock solution of endostatin at 7.9 mg ml⁻¹ in the buffer as supplied by the manufacturer, is kept at ice or at room temperature, readily aggregates. Perhaps, during the course of our experiments, part of the endostatin molecules start to denature, giving rise to the observed binding to Fn F4-5. With this chip, interaction between Fn F4-5 and the protein ligands could be abolished simply by increasing the NaCl concentration from 140 mM to 1 M. This shows that the interaction was primarily based on charge interactions.

Our surface plasmon resonance data show that F domains expressed in S. cerevisiae can bind to polypeptides with a cross-β structure. Furthermore, the data show that both 0.1 M H₃PO₄ buffer pH 1.0 and 10 mM HEPES pH 7.4, 1 M NaCl, 1.5 mM CaCl₂, 10 mM εACA, 0.05% Tween-20 are suitable buffers to release polypeptides with a cross-β structure from cross-β structure-binding compounds. These buffers are also suitable to release cross-β structure-binding compounds and proteins that are bound to a ligand with a cross-β structure. These data are helpful during the design of a method to deplete solutions from cross-β structure rich compounds by using cross-β structure-binding polypeptides that are immobilized on a suitable supporting material.

### Example 2

Protein Solutions Contain Protein Aggregates with Cross-β Structure

Structural Analysis of Proteins in Solution

We analyzed a series of protein solutions that are used as therapeutics for human use for the presence of cross-β structures in the protein. Protein solutions were stored at −20° C., 4° C. (as recommended by the manufacturers), room temperature, 37° C. or 65° C. Fluorescence of Congo red and ThT in the presence or absence of the proteins was analyzed, as well as tPA binding, tPA activation and factor XII activation. For fluorescence assays, 10 μg ml⁻¹ Aβ(1-40) E22Q amyloid was used as a positive control and gave typical values of approximately 1250 and 1800 A.U., respectively in the Congo red- and ThT fluorescence assay. Furthermore, TEM images were recorded to get insight whether amorphous aggregates are formed or fibrillar like structures.

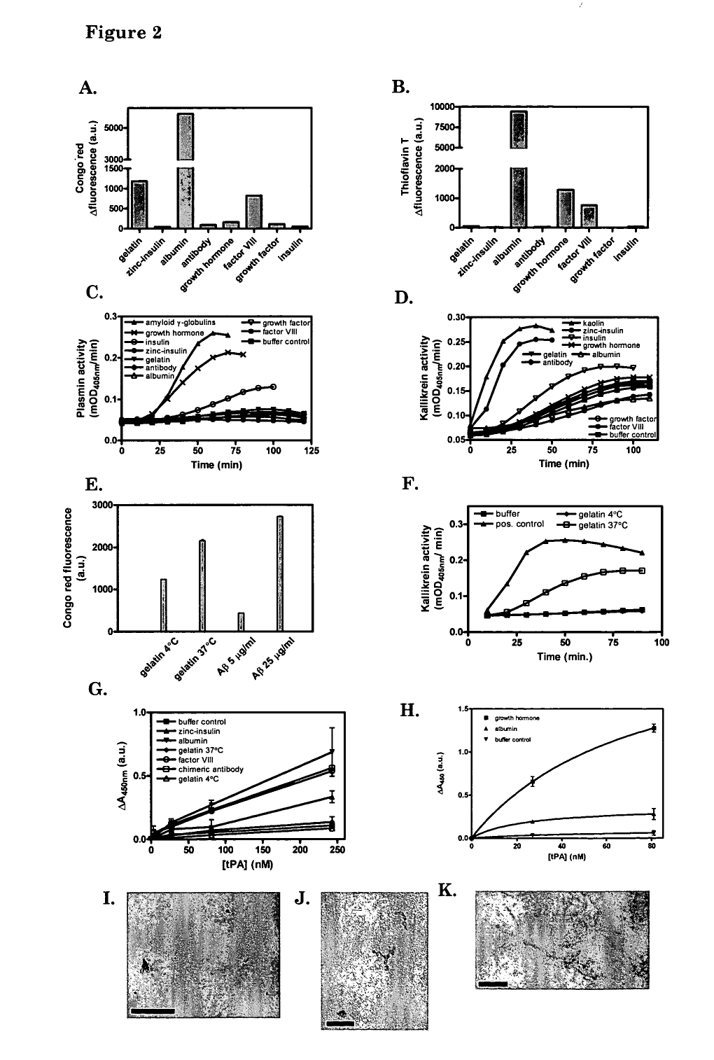

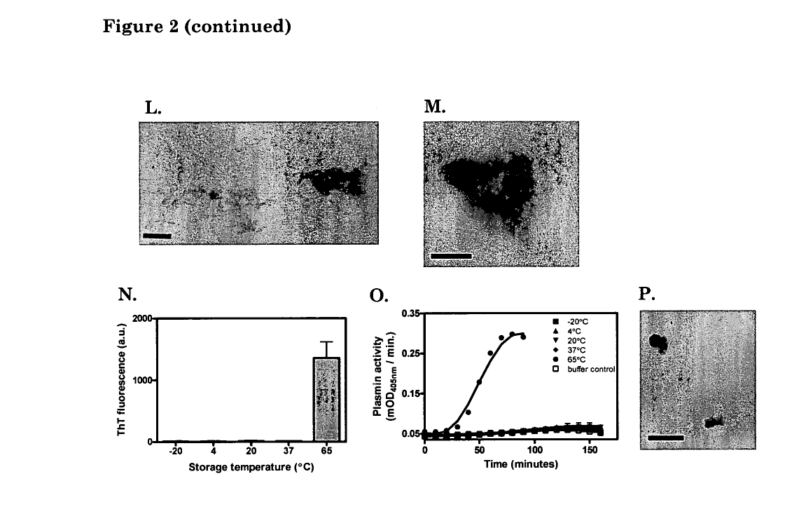

Gelatin, Cealb, FVIII and to some extent GH, stored at the recommended storage temperature of 4° C., enhanced the fluorescence of Congo red (FIG. 2, Panel A). In addition, Cealb, GH and FVIII enhance fluorescence of ThT (FIG. 2, Panel B). GH also induced tPA activation (FIG. 2, Panel C). Insulin activated tPA to a lesser extent, but still significantly (FIG. 2, Panel C). Both insulin and zinc-chelated insulin activate the factor XII/prekallikrein contact system (FIG. 2, Panel D). Gelatinous collagen fragments stored at 4° C. and 37° C. displayed enhanced Congo red fluorescence in a storage temperature dependent manner (FIG. 2, Panel E). Only gelatin kept at 37° C. activated factor XII (FIG. 2, Panel F). In an ELISA set-up, binding of tPA was established for Cealb, Reopro, gelatin, zinc-chelated insulin (FIG. 2, Panel G) and GH (FIG. 2, Panel H), all stored at the recommended temperature of 4° C. For both ELISAs, Hb-AGE was coated as a positive control (not shown for clarity). In the ELISA depicted in FIG. 2, Panel G, truncated K2P-tPA, or reteplase, which lacks the amyloid-binding F domain, was also tested for binding to the immobilized protein therapeutics. K2P-tPA did not bind to any of the therapeutics tested (not shown). On TEM images various condensed aggregates are seen with modified gelatin (FIG. 2, Panel I). GH appeared on TEM images as linear, branched and condense particles, all apparently composed of spherical particles (FIG. 2, Panel J). Zinc-chelated insulin appears on TEM images as thin linear unbranched fibrils with varying length (FIG. 2, Panel K). FVIII and Reopro did not appear as visible particles under the electron microscope. Cealb and insulin appeared as visible aggregates with no sign of a fibrillar nature (FIG. 2, Panels L, M). Reopro displayed storage temperature dependent ThT fluorescence enhancement properties and tPA activating properties (FIG. 2, Panels N, O). Only after storage at 65° C. ReoPro enhanced ThT fluorescence and induced Pls activity. Apparently, only at 65° C. ReoPro adopts the amyloid-like cross-β structure conformation. A TEM image of ReoPro that was stored at the recommended temperature of 4° C. revealed that some non-fibrillar aggregates were present, that apparently do not have ThT fluorescence enhancing or tPA activating properties under the conditions tested.

Protein Solutions Display Amyloid-Like Characteristics

Based on the observed binding of Congo red, ThT and tPA, based on the appearance on TEM images, and based on the observed activating properties towards tPA and factor XII, the tested solutions of Cealb, gelatin, insulin, zinc-insulin, GH, Reopro and FVIII displayed amyloid-like properties, when stored under recommended conditions. For Cealb, binding of tPA, Congo red and ThT is indicative for the presence of a cross-β structure. Binding of Congo red and activation of factor XII indicate the presence of cross-β structure conformation in gelatin. Binding of ThT and tPA, and activation of tPA by GH are indicative for amyloid-like properties in this solution. Finally, both activation of tPA and factor XII by insulin/zinc-insulin are indicative for the presence of cross-β structure rich aggregates. These data show the presence of protein or peptide aggregates with amyloid-like properties or the potential that the cross-β structure can be formed upon storage in these protein solutions.

Structural analysis of the tested proteins can be expanded using techniques and assays such as X-ray diffraction experiments, Fourier transform infrared spectroscopy, size exclusion HPLC, CD spectropolarimetry and binding assays using amyloid binding proteins, and can be expanded by introducing new protein solutions in the series of analyses.

### Example 3

Structure Analysis of Various P₂-glycoprotein I Preparations

Factor XII and tPA Bind to Recombinant β₂GPI and to β₂GPI Purified from Frozen Plasma, but not to β₂GPI Purified from Fresh Plasma

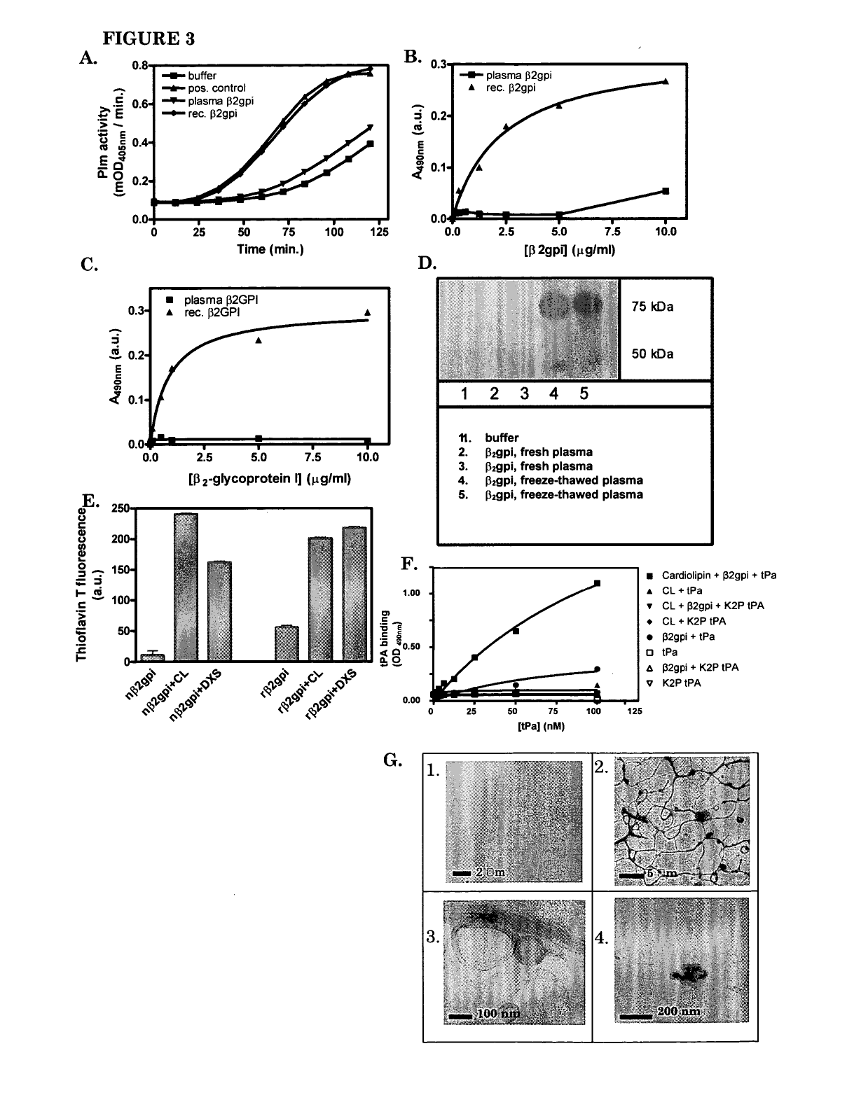

Recombinant β₂GPI, but not β₂GPI purified from fresh plasma stimulate tPA-mediated conversion of Plg to Pls, as measured as the conversion of the Pls-specific chromogenic substrate S-2251 (FIG. 3, Panel A). An ELISA demonstrated that tPA and factor XII bind recombinant β₂GPI, but not to β₂GPI purified from fresh human plasma (FIG. 3, Panels B, C). Recombinant β₂GPI binds to factor XII with a k_{D} of 20 nM (FIG. 3, Panel C) and to tPA with a k_{D} of 51 nM (FIG. 3, Panel B). In addition, factor XII co-elutes from the anti-β₂GPI antibody affinity column with β₂GPI, which was purified from plasma that was frozen at −20° C. and subsequently thawed, as shown on Western blot after incubation of the blot with anti-factor XII antibody (FIG. 3, Panel D). This shows that β₂GPI refolds into a conformation containing cross-β structures upon freezing. FIG. 3, Panel F shows that exposure of β₂GPI to CL or DXS500k introduces an increased ThT fluorescence signal, indicative for a conformational change in β₂GPI accompanied with the formation of cross-β structure conformation. Again, recombinant β₂GPI initially already gave a higher ThT fluorescence signal than native β₂GPI purified from plasma. These data not only show that recombinant β2GPI already comprises more cross-β structure conformation than plasma β2GPI, but that recombinant β2GPI also adopts more readily this conformation when environmental factors change. In FIG. 3, Panel G it is shown that exposure of β₂GPI to CL, immobilized on the wells of an ELISA plate, renders β₂GPI with tPA binding capacity. Binding of β₂GPI directly to the ELISA plate results in less tPA binding. These observations also show that CL has a denaturing effect, thereby inducing amyloid-like conformation in β₂GPI, necessary for tPA binding. These observations, together with the observation that exposure of β₂GPI to CL vesicles induced ThT binding capacity (FIG. 3, Panel F), show that exposure of β₂GPI to a denaturing surface induces formation of amyloid-like cross-β structure conformation. Furthermore, large fibrillar structures are seen on TEM images of plasma β₂GPI in contact with CL (FIG. 3, Panel G, image 2 and 3). Small CL vesicles seem to be attached to the fibrillar β2GPI. Images of plasma β₂GPI alone (FIG. 3, Panel G, image 1) or CL alone (not shown) revealed that no visible ultrastructures are present. In contrast, non-fibrillar aggregates and relatively thin curly fibrils can be seen on images of recombinant β₂GPI (FIG. 7, Panel G, image 4). These observation show that exposure of β₂GPI to CL and expression and purification of recombinant β₂GPI result in an altered multimeric structure of β₂GPI, when compared to the monomeric structure observed with X-ray crystallography.²⁴ Exposure of β₂GPI to CL or DXS500k induces an increased fluorescence when ThT is added, indicative for the formation of cross-β structure conformation when β₂GPI contacts a negatively charged surface. We predict that the cross-β structure can be relatively easily formed by one or more of the five domains of the extended β₂GPI molecule.²⁴ Each domain comprises at least one β-sheet that may function as a seed for local refolding into a cross-β structure.

In conclusion, it is revealed that several physical/chemical conditions influence the fold of the protein. Exposure to CL or DXS500, a freeze-thaw cycle, variations in protein purification protocol, the source of the protein and exposure to plastic all introduce a structural rearrangement in the protein accompanied by the formation of the amyloid-like cross-β structure fold. This new fold can be detected by, amongst others, tPA binding, tPA activation, factor XII binding and by conventional amyloid fluorescence assays.

### Example 4

Induction of Cross-β Structures in Proteins

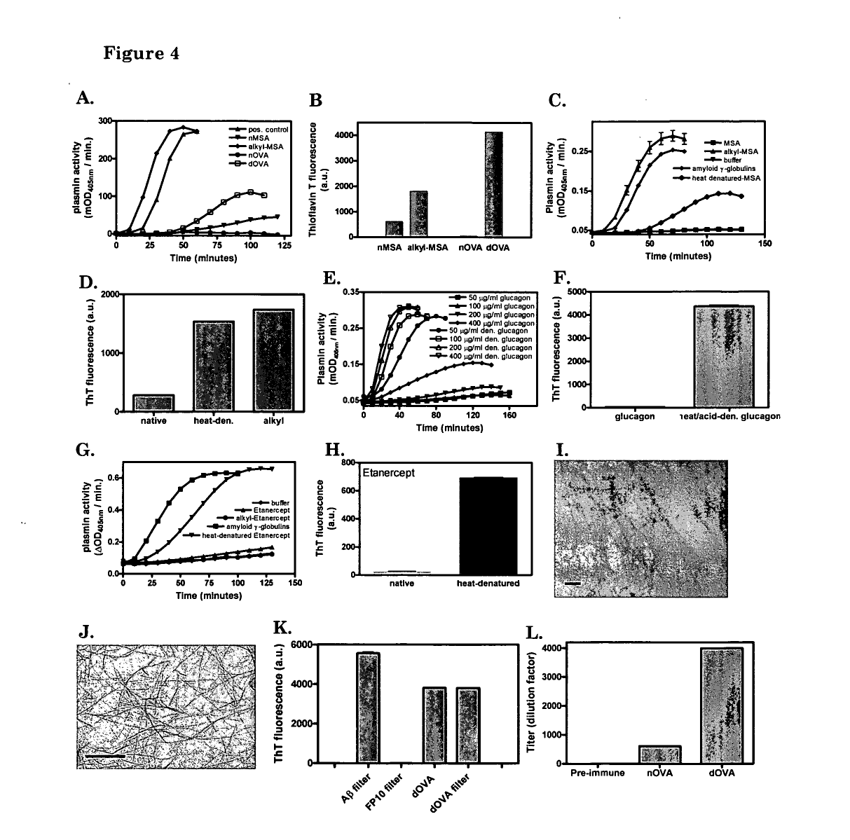

OVA with amyloid-like properties was obtained by heat denaturation at 85° C. (FIG. 4, Panels A, B, I, K). The presence of cross-β structures was established with ThT fluorescence and Plg-activation assays and by TEM imaging. The fibrillar structures of at least up to 2 μm in length, seen on the TEM images are likely not the only OVA assemblies with cross-β structures present, as concluded from the observation that filtration through a 0.2 μm filter does not reduce the enhancement of ThT fluorescence. A person skilled in the art can perform similar experiments with murine serum albumin (MSA), human glucagon or Etanercept, such as those described below (FIG. 4).

The amyloid-like protein fold was induced in MSA by heat denaturation at 85° C. and by reduction and alkylation of disulphide bonds (FIG. 4, Panels A-D). We observed that also native MSA enhanced ThT fluorescence to some extent, but this was not reflected by stimulation of tPA activation. Although heat-denatured MSA and alkylated MSA enhance ThT fluorescence to a similar extent, they differ in tPA activating potential. This suggests that tPA and ThT interact with distinct aspects of the cross-β structure. Previously, we observed that Congo red, another amyloid-specific dye, can efficiently compete for tPA binding to amyloid-like aggregates in ELISAs, whereas ThT did not inhibit tPA binding at all (patent application P57716EP00 and B.Bouma, unpublished data).

Amyloid-like cross-β structure conformation was induced in glucagon by heat-denaturation at 37° C. at low pH in HCl buffer (FIG. 4, Panels E, F, J). In this way, a potent activator of tPA was obtained, that enhanced ThT fluorescence to a large extent. In addition, long and bended unbranched fibrils were formed, as visualized on TEM images (FIG. 4, Panel J). Noteworthy, at high glucagon concentration, also native glucagon had some tPA activating potential, indicative for the presence of a certain amount of cross-β structure rich protein.

Alkylated Etanercept did not activate tPA at all, whereas heat-denatured Etanercept had similar tPA activating potential as amyloid γ-globulins (FIG. 4, Panel G). After heat denaturation, Etanercept also efficiently induced enhanced ThT fluorescence (FIG. 4, Panel H). Native Etanercept both induced some tPA activation and gave some ThT fluorescence enhancement.

From our analyses we conclude that dOVA, alkyl-MSA, heat/acid-denatured glucagon and heat-denatured Etanercept comprise the cross-β structure conformation. The presence of the cross-β structures can be further established by circular dichroism spectropolarimetry analyzes, X-ray fiber diffraction experiments, Fourier transform infrared spectroscopy, Congo red fluorescence/birefringence, tPA binding, factor XII activation and binding, and more.

### REFERENCE LIST

1. Cleland J. L., M. F. Powell and S. J. Shire. The development of stable protein formulations: a close look at protein aggregation, deamidation, and oxidation. Crit Rev. Ther. Drug Carrier Syst. 10, 307-377 (1993).
2. Wang W. Protein aggregation and its inhibition in biopharmaceutics. Int. J Pharm. 289, 1-30 (2005).
3. Krishnamurthy R. and M. C. Manning. The stability factor: importance in formulation development. Curr. Pharm. Biotechnol. 3, 361-371 (2002).
4. Hermeling S., D. J. Crommelin, H. Schellekens and W. Jiskoot. Structure-immunogenicity relationships of therapeutic proteins. Pharm. Res. 21, 897-903 (2004).
5. Bouma B. et al. Glycation induces formation of amyloid cross-beta structure in albumin. J. Biol. Chem. 278, 41810-41819 (2003).
6. Nilsson M. R. Techniques to study amyloid fibril formation in vitro. Methods 34, 151-160 (2004).
7. Bucciantini M. et al. Inherent toxicity of aggregates implies a common mechanism for protein misfolding diseases. Nature 416, 507-511 (2002).
8. Cribbs D. H., B. Y. Azizeh, C. W. Cotman and F. M. LaFerla. Fibril formation and neurotoxicity by a herpes simplex virus glycoprotein B fragment with homology to the Alzheimer's A beta peptide. Biochemistry 39, 5988-5994 (2000).
9. Kayed R. et al. Common structure of soluble amyloid oligomers implies common mechanism of pathogenesis. Science 300, 486-489 (2003).
10. Kranenburg O. et al. Recombinant endostatin forms amyloid fibrils that bind and are cytotoxic to murine neuroblastoma cells in vitro. FEBS Lett. 539, 149-155 (2003).
11. Tucker H. M., M. Kihiko-Ehmann, S. Wright, R. E. Rydel and S. Estus. Tissue plasminogen activator requires plasminogen to modulate amyloid-beta neurotoxicity and deposition. J. Neurochem. 75, 2172-2177 (2000).
12. Klein W. L., G. A. Krafft, and C. E. Finch. Targeting small Abeta oligomers: the solution to an Alzheimer's disease conundrum? Trends Neurosci. 24, 219-224 (2001).
13. Buxbaum J. N. Diseases of protein conformation: what do in vitro experiments tell us about in vivo diseases? Trends Biochem. Sci. 28, 585-592 (2003).
14. Reixach N., S. Deechongkit, X. Jiang, J. W. Kelly and J. N. Buxbaum. Tissue damage in the amyloidoses: Transthyretin monomers and nonnative oligomers are the major cytotoxic species in tissue culture. Proc. Natl. Acad. Sci. U. S. A 101, 2817-2822 (2004).
15. O'Nuallain B. and R. Wetzel. Conformational Abs recognizing a generic amyloid fibril epitope. Proc. Natl. Acad. Sci. U. S. A 99, 1485-1490 (2002).
16. Kranenburg O. et al. Tissue-type plasminogen activator is a multiligand cross-beta structure receptor. Curr. Biol. 12, 1833-1839 (2002).
17. Schielen J. G., H. P. Adams, M. Voskuilen, G. J. Tesser and W. Nieuwenhuizen. Structural requirements of position A alpha-157 in fibrinogen for the fibrin-induced rate enhancement of the activation of plasminogen by tissue-type plasminogen activator. Biochem. J. 276, 655-659 (1991).
18. Thomassen Y. E., W. J. Meijer, L. Sierkstra and C. T. Verrips. Large-scale production of VHH antibody fragments by Saccharomyces cerevisiae. Enzyme and Microbial. Technology 30, 273-278 (2002).
19. Hackeng T. M., J. H. Griffin and P. E. Dawson. Protein synthesis by native chemical ligation: expanded scope by using straightforward methodology. Proc. Natl. Acad. Sci. U. S. A 96, 10068-10073 (1999).
20. de Laat B., R. H. Derksen, R. T. Urbanus and P. G. de Groot. IgG antibodies that recognize epitope Gly40-Arg43 in domain I of {beta}2-glycoprotein I cause LAC and their presence correlates strongly with thrombosis. Blood, (2004).
21. Horbach D. A., E. van Oort, R. C. Donders, R. H. Derksen and P. G. de Groot. Lupus anticoagulant is the strongest risk factor for both venous and arterial thrombosis in patients with systemic lupus erythematosus. Comparison between different assays for the detection of antiphospholipid antibodies. Thromb. Haemost. 76, 916-924 (1996).
22. Horbach D. A., E. van Oort, M. J. Tempelman, R. H. Derksen and P. G. de Groot. The prevalence of a non-phospholipid-binding form of beta2-glycoprotein I in human plasma-consequences for the development of anti-beta2-glycoprotein I antibodies. Thromb. Haemost. 80, 791-797 (1998).
23. Onoue S. et al. Mishandling of the therapeutic peptide glucagon generates cytotoxic amyloidogenic fibrils. Pharm. Res. 21, 1274-1283 (2004).
24. Bouma B. et al. Adhesion mechanism of human beta(2)-glycoprotein I to phospholipids based on its crystal structure. EMBO J. 18, 5166-5174 (1999).

1. A method for detecting a protein and/or peptide comprising a cross-β structure in an aqueous solution comprising a protein, the method comprising:
a. contacting the aqueous solution with at least one cross-β structure-binding compound resulting in a bound protein and/or peptide comprising a cross-β structure, and
b. detecting whether bound proteins and/or peptides comprising a cross-β structure are present in the aqueous solution.

2. The method according to claim 1, wherein said cross-β structure-binding compound is a compound according to Table 1, Table 2, or Table 3.

3. The method according to claim 1, wherein the aqueous solution further comprises a material selected from the group consisting of a detergent, a food, a food supplement, a cell culture medium, a commercially available protein, protein/peptide solutions used for research purposes, blood, blood products, a cosmetic product, a cell, and a combination of any thereof.

4. The method according to claim 2, wherein the aqueous solution further comprises a material selected from the group consisting of a detergent, a food, a food supplement, a cell culture medium, a commercially available protein, protein/peptide solutions used for research purposes, blood, blood products, a cosmetic product, a cell, and a combination of any thereof.

5. (canceled)

6. A method for removing a protein and/or peptide comprising a cross-β structure from an aqueous solution comprising a protein, said method comprising:
a. contacting the aqueous solution with at least one cross-β structure-binding compound resulting in a bound protein and/or peptide comprising a cross-β structure,
b. binding said protein and/or peptide comprising a cross-β structure to said cross-β structure-binding compound, and,
c. separating said bound protein and/or peptide comprising a cross-β structure from the aqueous solution.

7. The method according to claim 6, wherein said cross-β structure-binding compound is a compound according to Table 1, Table 2, or Table 3.

8. The method according to claim 6, wherein said cross-β binding compound is bound to a second compound.

9. (canceled)

10. The method according to claim 7, wherein said cross-β binding compound is bound to a second compound.

11. The method according to claim 10, wherein said second compound is bound to a solid phase.

12. A method for decreasing and/or preventing undesired side effects of an aqueous solution comprising a protein and/or increasing the specific activity per gram protein of an aqueous solution, said method comprising:
detecting any unfolded protein or peptide and/or aggregated protein or peptide and/or multimerized protein or peptide comprising a cross-β structure in the aqueous solution by a detection method comprising contacting the aqueous solution with at least one cross-β structure-binding compound resulting in a bound protein and/or peptide comprising a cross-β structure, and detecting whether bound proteins and/or peptides comprising a cross-β structure are present in the aqueous solution, and
removing any unfolded protein or peptide and/or aggregated protein or peptide and/or multimerized protein or peptide comprising a cross-β structure from the aqueous solution with a method comprising:
contacting the aqueous solution with at least one cross-β structure-binding compound resulting in a bound protein and/or peptide comprising a cross-β structure, binding said protein and/or peptide comprising a cross-β structure to said cross-β structure-binding compound, and,
separating said bound protein and/or peptide comprising a cross-β structure from the aqueous solution.

13. An aqueous solution comprising a protein, obtainable by the method according to of claim 6.

14. A kit for use with an aqueous solution suspected to contain a peptide comprising a cross-β structure, said kit comprising:
a. at least one cross-β structure-binding compound to bind a peptide comprising a cross-β structure present in the aqueous solution, and
b. means for detecting whether a peptide comprising a cross-β structure bonded to the at least one cross-β structure-binding compound is present in the aqueous solution.

15. A method for detecting a cell comprising a protein and/or peptide with cross-β structure on its surface in a collection of cells, said method comprising:
contacting said cell with a cross-β structure-binding molecule, and
measuring binding of said molecule to said cell so as to detect a cell comprising the protein and/or peptide with cross-beta structure on its surface.

16. A method for removing a cell comprising a protein and/or peptide with cross-β structure on its surface from a collection of cells, said method comprising:
contacting said cell with a cross-β structure-binding molecule, and
binding said molecule to a solid surface so as to enable removal of a cell comprising the protein and/or peptide with cross-beta structure on its surface.

17. An aqueous solution comprising a protein, obtainable by the method according to of claim 12.

18. An aqueous solution comprising a protein, obtainable by the method according to of claim 7.

19. An aqueous solution comprising a protein, obtainable by the method according to of claim 8.

20. An aqueous solution comprising a protein, obtainable by the method according to claim 9.

21. The method according to claim 1 wherein the cross-β structure-binding compound is a finger domain.

22. A method for detecting a protein and/or peptide comprising a cross-β structure in an aqueous solution comprising a protein, the method comprising:
a. contacting the aqueous solution with means for binding to polypeptides with cross-β structure, wherein said contacting results in a bound protein and/or peptide comprising a cross-β structure, and
b. detecting whether bound proteins and/or peptides comprising a cross-β structure are present in the aqueous solution.


SEQUENCE LISTING (5 sequences)

SEQ ID NO: 1 - protein, 13 aa, Artificial
  Amyloid fibrin peptide
KRLEVDIDIGIRS

SEQ ID NO: 2 - protein, 40 aa, Artificial
  Human amyloid-beta
DAEFRHDSGYEVHHQKLVFFAQDVGSNKGAIIGLMVGGVV

SEQ ID NO: 3 - protein, 10 aa, Artificial
  Non-amyloid fragment FP10 of human fibrin alpha-chain (148-157)
KRLEVDIDIK

SEQ ID NO: 4 - DNA, 28 bp, Artificial
  Forward primer comprising a BglII site
gagatctgctcaaaacatcacagcccgg

SEQ ID NO: 5 - DNA, 28 bp, Artificial
  Reverse primer with a NotI site
gcggccgcctcgcctggttcgatgatgc